SEQUENCE LISTING (29 sequences)

SEQ ID NO: 1 - DNA, 282 bp, Homo sapiens
  source 1..282: mol_type = genomic DNA
ggccgggcgcggtggctcacgcctgtaatcccagcactttgggaggccgaggcgggcggatcacctgaggtcaggagttcgagaccagcccggccaacacggtgaaaccccgtctctactaaaaatacaaaaattagccgggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctc

SEQ ID NO: 2 - DNA, 6065 bp, Homo sapiens
  source 1..6065: mol_type = genomic DNA
gagaggagccaagatggccgaatagcaacagctccggtctacagctcccagcgtgagcgacgcagaagacgggtgatttctgcatttccatctgaggtactgggttcatctcactagggagtgccagacagtgggcgcaggtcagtgggtgcgcgcaccgtgcacgagccgaagcagggcgaggcattgtctcacttgggaagcgcaaggggtcagggagttccctttccgagtcaaagaaaggggtgacggacgcacctggaaaatcgggtctctcccacccgaatattgcgctttcggaccggcttaaaaaacggcgcaccgcgagattatatcttgcacctggctaggagggtcctacgcccacggagtctcgctgattgctagcacagcagtctgagatcaaactgcaaggccgcagcaaggctgggggaggggcgcccgccattgcccaggcttgcttgggtaaacaaagcagcctggcagctcgaactgggtggagcccaccacagctcaaggaggcctgcctgcctttgtaggctccacctctgggggcagggcacagacaaataaaaagacagcagtaacctctgcagacttaaatgtccctgtctgacagctttgaagagagcagtggttctcccagcatgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaacagaaaggacatccacaccaaaaacccatctgtacatcaccatcatcaaagatcaaaagtagataaaaccacgaagatggggaaaaaacagaacagaaaaactggaaactgtaaaatgcatagtgcctctcctcctccaaaggaacacagttcctcaccagcaacggaacaaagctggatggagaatgactttgacgagctgagagaagaaggcttcagatgatcaaattactctgagctatgggaggacattcaaaccaaaggcaaagaagttgaaacctttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcaagaagggaagtttagagacaaaagaataaaaagaaatgagcaaagcctccaagaaatatgggactatgtgaaaagaccaaatctatgtctgatcggtgtacctgaaagtgatggggacaatggaaccaagttggaaaacacgctgcaggatattatccaggagaacttccccaatctagcaaggaaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaagggaaacccatcagactaacagcggatctctcagcagaaaccctacaagccagaagagagtgggggccaatattcaacattcttaaagaaaagaattttcaacccagaatttcatatccagccaaactaagcttcataagtgaaggagaaataaaatcctttacagacaagaaatgctgagagattttgtcaccaccaggcctaccctaaaagagctcctgaaggaagcgccaaacatggaaaggaacaaccggtaccagccgctgcaaaatcatgccaaaatgtaaagaccatcgagactaggaagaaactgcatcaactaacgagcaaaatcaccagctaacatcataatgacaggatcaaattcacacataacaatattaactttaaatgtaaatggattaaatgctccaattaaaagacacagactggcaaattggataaagagtcaagacccatcagtgtgctgtattcaggaaacccatctcatgtgcagagacacacataggctcaaaataaaaggatggaggaagatctaccaagcaaatgtaaaacaaaaaaaggcaggggttgcaatcctagtctctgataaaacagactttaaaccaacaaagatcaaaagagacaaagaaggccattacataatggtaaagggatcaattcaacaagaagagctaactatcctaaatatatatgcacccaatacaggagcacccagattcataaagaaagtcatgagtgacctacaaagagacttagactcccacacattaataatgggagactttaacaccccactgtcaacattagacagatcaacgagacagaaagtcaacaaggatacccaggaattgaacctctgcaccaagcagacctaatagacatctacagaactctccaccccaaatcaacagaatctacatttttttcagcaccacaccacacctattccaaaattgaccacatacttggaagtaaagctctcctcagcaaatgtaaaagaacagaaattataacaaactatctctcagaccacagtgcaatcaaactagaactcaggattaagaatctcattcaaaaccgctcaactacatggaaactgaacaacctgctcctgaatgactactgggtacataacgaaatgaaggcagaaataaagatgttctttgaaaccaacgagaacaaagacacaacataccagaatctctgggacgcattcaaagcagtgtgtagagggaaatttatagcactaaatggctacaagagaaagcaggaaagatccaaaattgacacccgaacatcacaattaaaagaactagaaaagcaagagcaaacacattcaaaagctagcagaaggcaagaaataactaaaatcagagcagaactgaaggaaatagagacacaaaaaacccttcaaaaaattaatgaatccaggagcttgttttttgaaaggatcaacaaaattgatagaccgctagcaagactaataaagaaaaaaagagagaagaatctaatagacacaataaaaaatgataaaggggatatcaccaccgatcccacagaaatacaaactaccatcagagaatactacaaacacctctactcaaataaactagaaaatctagaagaaatggataaattcctcgacacatacactctcccaagactaaaccaggaagaagttgaatctctgaatagaccaaaaacaggatctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaatgaatccagcagcacatcaaaaagtttatccaccgtgattaagtgggcttcatccctgggatgcaagactggttcaatatgcacaaatcaataaatgtaatccagcatataaacagaaccaaagacaaaaaccacatgattgtctcaatagatgcagaaaaagcctttgacaaaattcaacaacccttcatgctaaaaactctcaataaattaggtattgatgggacgtatttcaaaataataagagctatctatgacaaacccacagccaatatcatactgaatggtcaaaaactggaagcattccctttgaaaactggcacaagacagggatgccctctctcaccactcctattcaacatagtgttggaagttctggccagggcaattaggcaggagaaggaaataaagggtattcaattaggaaaagaggaagtcaaattgtccctgtttgcagacgacatgattgtatatctagaaaaccccattgtctcagcccaaaatctccttaagccgataagcaacttcagcaaagtctcaggatacaaaatcaatgtacaaaaatcacaagcattcttatacaccaacaacagacaaacagagagccaaatcatgaatgaactaccattcacaattgcttcaaagagaataaaatacttaggaatccaaattacaagggatgtgaaggacctcttcaaggagaactacaaaccactgctcaaggaaataaaagaggatacaaacaaatggaagaacattccatgctcatgggtaggaagaatcaatatcatgaaaatggccatactgcccaaggtaatttacagattcaataccatccccataaagctaccaatgactttcttcacagaattggaaaaaactactttaaagttcatatggaaccaaaaaagggcccgcattgccaaggcaatccgaagccaaaagaacaaatctggaggcatcacactacctgacttcaaactatactacaaggctacagtaaccaaaacagcatggtactggtaccaaaacagagatatagatcaatggaacagaacagagccctcagaaataacgccgcatatctacaactatctgatctttgacaaacctgagaaaaatgagcaatggggaaaggattccctatttaataaatggtgctgggaaaactggctagccatatgtagaaagctgaaactggatcctttccttacaccttatacaaaaatcaattcaagatggattaaagacttaatcgttagacctaaaaccataaaaaccctagaagaaaacctaggcattaccattcaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaggcaacctacaaaatgggagaaaactttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaaaaaatgctcaccatcactggccatcagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactagttcaaccgttgtggaagtcagtgtggcgattcctcagggatctagaaccagaaataccatttgacccagccatcccattactgggtatatacccaaaggactctaaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcgtgtcctttgtagggacatggatgaaattggaaatcatcattcttagtaaactatcgcaagaacaaaaaaccaaacactgcatattctcactcataggtgggaattgaacaatgagatcacatggacacaggaaggggaatatcacactctggggactgtggtggggtggggggagaggggagggatagcattgggagatatacctaatgctagatgacgagttagtgggtgcagtgcaccagcatggcacatttatacatatgtaactaacctgcacaatgtgcacatgtaccctaaaacttaaagtataataaaaaaaaataaaaaaaataaaaataaaaaagaaaatatgggaaaggtaaaaaa

SEQ ID NO: 3 - DNA, 128 bp, synthetic construct
  source 1..128: mol_type = other DNA
gccaagatggccgaataggaacagctccggtctacagctcccagcgtgagcgacgcagaagacgggtgatttctgcatttccatctgaggtaccgggttcatctcactagggagtgccagacagtggg

SEQ ID NO: 4 - DNA, 101 bp, synthetic construct
  source 1..101: mol_type = other DNA
gacagtgggcgcaggccagtgtgtgtgcgcaccgtgcgcgagccgaagcagggcgaggcattgcctcacctgggaagcgcaaggggtcagggagttccctt

SEQ ID NO: 5 - DNA, 108 bp, synthetic construct
  source 1..108: mol_type = other DNA
caaggggtcagggagttccctttccgagtcaaagaaaggggtgacggacgcacctggaaaatcgggtcactcccacccgaatattgcgcttttcagaccggcttaaga

SEQ ID NO: 6 - DNA, 121 bp, synthetic construct
  source 1..121: mol_type = other DNA
ggaatctcgctgattgctagcacagcagtctgagatcaaactgcaaggcggcaacgaggctgggggaggggcgcccgccattgcccaggcttgcttaggtaaacaaagcagccgggaagct

SEQ ID NO: 7 - DNA, 130 bp, synthetic construct
  source 1..130: mol_type = other DNA
aagtgcttaaaggagctgatggagctgaaaaccaaggctcgagaactacgtgaagaatgcagaagcctcaggagccgatgcgatcaactggaagaaagggtatcagcaatggaagatgaaatgaatgaaa

SEQ ID NO: 8 - DNA, 120 bp, synthetic construct
  source 1..120: mol_type = other DNA
tgaaggaaaaaatgttaagggcagccagagagaaaggtcgggttaccctcaaaggaaagcccatcagactaacagcggatctctcggcagaaaccctacaagccagaagagagtgggggc

SEQ ID NO: 9 - DNA, 150 bp, synthetic construct
  source 1..150: mol_type = other DNA
tggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaattt

SEQ ID NO: 10 - DNA, 125 bp, synthetic construct
  source 1..125: mol_type = other DNA
tgagttcatatcctttgtagggacatggatgaaattggaaaccatcattctcagtaaactatcgcaagaacaaaaaaccaaacaccgcatattctcactcataggtgggaattgaacaatgagat

SEQ ID NO: 11 - DNA, 63 bp, synthetic construct
  source 1..63: mol_type = other DNA
acactgacgacatggttctacannnnnnnnnnnnnnnngttaagatggtygaataggaatagt

SEQ ID NO: 12 - DNA, 62 bp, synthetic construct
  source 1..62: mol_type = other DNA
tacggtagcagagacttggtctnnnnnnnnnnnnnnnncccactatctaacactccctaata

SEQ ID NO: 13 - DNA, 60 bp, synthetic construct
  source 1..60: mol_type = other DNA
acactgacgacatggttctacannnnnnnnnnnnnnnngatagtgggygtaggttagtgt

SEQ ID NO: 14 - DNA, 61 bp, synthetic construct
  source 1..61: mol_type = other DNA
tacggtagcagagacttggtctnnnnnnnnnnnnnnnnaaaaaaactccctaaccccttac

SEQ ID NO: 15 - DNA, 62 bp, synthetic construct
  source 1..62: mol_type = other DNA
acactgacgacatggttctacannnnnnnnnnnnnnnntaaggggttagggagttttttttt

SEQ ID NO: 16 - DNA, 66 bp, synthetic construct
  source 1..66: mol_type = other DNA
tacggtagcagagacttggtctnnnnnnnnnnnnnnnntcttaaaccratctaaaaaacrcaatat

SEQ ID NO: 17 - DNA, 65 bp, synthetic construct
  source 1..65: mol_type = other DNA
acactgacgacatggttctacannnnnnnnnnnnnnnnggaatttygttgattgttagtatagta

SEQ ID NO: 18 - DNA, 62 bp, synthetic construct
  source 1..62: mol_type = other DNA
tacggtagcagagacttggtctnnnnnnnnnnnnnnnnaacttcccractactttatttacc

SEQ ID NO: 19 - DNA, 63 bp, synthetic construct
  source 1..63: mol_type = other DNA
acactgacgacatggttctacannnnnnnnnnnnnnnnaagtgtttaaaggagttgatggagt

SEQ ID NO: 20 - DNA, 64 bp, synthetic construct
  source 1..64: mol_type = other DNA
tacggtagcagagacttggtctnnnnnnnnnnnnnnnntttcattcatttcatcttccattact

SEQ ID NO: 21 - DNA, 63 bp, synthetic construct
  source 1..63: mol_type = other DNA
acactgacgacatggttctacannnnnnnnnnnnnnnntgaaggaaaaaatgttaagggtagt

SEQ ID NO: 22 - DNA, 61 bp, synthetic construct
  source 1..61: mol_type = other DNA
tacggtagcagagacttggtctnnnnnnnnnnnnnnnnacccccactctcttctaacttat

SEQ ID NO: 23 - DNA, 66 bp, synthetic construct
  source 1..66: mol_type = other DNA
acactgacgacatggttctacannnnnnnnnnnnnnnntggaaattgaataatttgtttttgaatg

SEQ ID NO: 24 - DNA, 64 bp, synthetic construct
  source 1..64: mol_type = other DNA
tacggtagcagagacttggtctnnnnnnnnnnnnnnnnaaatttccctctacacactactttaa

SEQ ID NO: 25 - DNA, 65 bp, synthetic construct
  source 1..65: mol_type = other DNA
acactgacgacatggttctacannnnnnnnnnnnnnnntgagtttatattttttgtagggatatg

SEQ ID NO: 26 - DNA, 63 bp, synthetic construct
  source 1..63: mol_type = other DNA
tacggtagcagagacttggtctnnnnnnnnnnnnnnnnatctcattattcaattcccacctat

SEQ ID NO: 27 - DNA, 22 bp, synthetic construct
  source 1..22: mol_type = other DNA
tacggtagcagagacttggtct

SEQ ID NO: 28 - DNA, 22 bp, synthetic construct
  source 1..22: mol_type = other DNA
acactgacgacatggttctaca

SEQ ID NO: 29 - DNA, 6064 bp, Homo sapiens
  source 1..6064: mol_type = genomic DNA
gggaggaggagccaagatggccgaataggaacagctccggtctacagctcccagcgtgagcgacgcagaagacgggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgctgagagattttgtcaccaccaggcctgccctaaaagagctcctgaaggaagcgctaaacatggaaaggaacaaccggtaccagccgctgcaaaatcatgccaaaatgtaaagaccatcgagactaggaagaaactgcatcaactaatgagcaaaatcaccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcatcgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttagacctaaaaccataaaaaccctagaagaaaacctaggcattaccattcaggacataggcgtgggcaaggacttcatgtccaaaacaccaaaagcaatggcaacaaaagccaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacgttagtgggtgcagcgcaccagcatggcacatgtatacatatgtaactaacctgcacaatgtgcacatgtaccctaaaacttagagtataataaaaaaaaaaaaaaaaaaaaaaaaaaaaaaaaaataaataaaaa

(19) United States
(12) Patent Application Publication
(10) Pub. No.: US 2026/0088128 A1
(43) Pub. Date: Mar. 26, 2026

(54) SENSITIVE AND SPECIFIC DETERMINATION OF DNA METHYLATION PROFILES

(71) Applicants: INSTITUT CURIE, Paris (FR); INSERM (INSTITUT NATIONAL DE LA SANTÉ ET DE LA RECHERCHE MÉDICALE), Paris (FR); CENTRE NATIONAL DE LA RECHERCHE SCIENTIFIQUE, Paris (FR); ECOLE NATIONALE SUPERIEURE DES MINES DE PARIS, Paris (FR); PARIS SCIENCES ET LETTRES, Paris (FR); SORBONNE UNIVERSITE, Paris (FR)

(72) Inventors: Charlotte PROUDHON, Rennes (FR); Chloé-Agathe AZENCOTT, Paris Cedex 6 (FR); Marc MICHEL, Paris (FR); Maryam HEIDARY, Paris (FR)

(21) Appl. No.: 19/107,390

(22) PCT Filed: Sep. 1, 2023
(86) PCT No.: PCT/EP2023/074092

§ 371 Date: Feb. 27, 2025

(30) Foreign Application Priority Data
Sep. 2, 2022 (WO) PCT/FR2022/000078
Dec. 21, 2022 (EP) 22306972.5

(51) Int. Cl. G16B 30/10; C12Q 1/6886; G16B 40/20

(52) U.S. Cl. CPC G16B 30/10; C12Q 1/6886; G16B 40/20; C12Q 2600/154

(57) ABSTRACT

The invention relates to methods for determining the methylation profile of DNA sequences of interest and methods for accurately distinguishing between a healthy methylation profile and a cancerous methylation profile, as well as to kits to implement them.

### FIELD OF THE INVENTION

The present invention relates to the field of medicine. It particularly concerns methods for determining the methylation profile of DNA sequences of interest and methods for accurately distinguishing between a healthy methylation profile and a cancerous methylation profile, as well as to kits to implement them.

### BACKGROUND OF THE INVENTION

The discovery of the mutations responsible for the initiation and progression of human cancers has revealed a new generation of biomarkers. These genetic biomarkers now help to determine the most suitable treatments and allow the development of targeted treatment regimens, recognized to improve clinical results. Current methods for identifying specific mutations in personalized medicine involve the use of tissue biopsies. However, the biopsy procedure is invasive, painful, carries some risks and is sometimes not feasible. It is also biased in space, thus failing to capture tumor heterogeneity, and in time as repetitive biopsies are usually not performed.

An attractive complement or alternative to tissue biopsies are liquid biopsies. Extensive research has shown that the tumor genetic alterations can be detected from the plasma of patients suffering from cancer. This paved the way to the implementation of molecular analyses carried out on liquid biopsies to genotype tumors in a non-invasive way and made it possible to demonstrate the potential of circulating tumor DNA (ctDNA) as a marker of cancer progression. Recent advances have demonstrated the benefits of ctDNA quantification, including the possibility to perform large-scale tumor genotyping, since ctDNA is derived from all tumor sites in the body. It is moreover a powerful prognostic factor enabling detection of tumor masses that are not clinically perceptible, after surgery or during treatment. These approaches promise optimal management of cancer patients and currently play an important role in oncology.

However, several technological hurdles still limit their large-scale use. Samples collected at early stages of tumor progression, or during and after treatment, may contain less than one mutant copy per milliliter of plasma. This is below the detection limit of most used technologies, even when testing multiple genetic mutations simultaneously. Moreover, most methods are biased towards preselected recurrent mutations which do not cover all tumors. Inventors have observed in their studies that approximately 25% of breast cancer patients do not have common mutations traceable in plasma DNA, even in advanced stages. The sensitivity of methods targeting genetic alterations remains limited by the low number of recurrent mutations detectable per genome. It is therefore necessary to develop new detection tools that are more sensitive and also more informative.

Multiple studies have demonstrated the central role of epigenetic processes in the onset, progression, and treatment of cancer. Epigenetic alterations (i.e., changes in the pattern of chromatin modifications such as DNA methylation and histone modifications) are promising candidates for the detection, diagnosis and prognosis of cancer. These markers provide an additional level of information, neglected by methods that only question genetic alterations. Unlike point mutations which affect only a single base pair per genome, epigenetic alterations are dispersed throughout the genome and affect multiple residues per region. Thus, new diagnostic strategies integrating epigenetic biomarkers would achieve increased sensitivity, but also cover cases without detectable mutations. Because the epigenetic landscape is highly cell-type specific, epigenetic markers can also inform about the tissue of origin of tumors. This is not the case for oncogenic mutations which are often common to several types of cancer. Epigenetic markers may be decisive in detecting early stages—when chances of recovery are the best—, residual disease, early stages of relapse, or in the acquisition of resistance during treatment. This will allow better monitoring of cancerous diseases and offer new therapeutic windows to treat and cure.

Aberrant DNA methylation is a hallmark of neoplastic cells, which combine hypermethylation of a wide range of tumor suppressor genes along with a global hypomethylation of the genome. DNA methylation is a stable modification, which affects a large number of CpG sites per region and per genome. Moreover, the concordance in methylation state between multiple CpGs from the same region can help detect low-frequency anomalies among a heterogeneous population of molecules. Finally, combining several genomic regions allows to capture a wide range of tumor alleles and to cover the heterogeneous profiles of cancer patients.

Until now, most studies investigating plasma DNA methylation patterns have targeted a small number of regions at high depth, using PCR-based methods, or explored the whole genome at low depth with high throughput sequencing. Both approaches have limited sensitivity. More recent studies, based on the capture of regions of interest coupled with deep sequencing have investigated the performance of a greater number of regions at high depth. These methods have enabled sensitive detection and classification of cancer from plasma DNA. However, since they largely focus on cancer hypermethylation and unique sequences, they involve targeting specific regions for each cancer subtype. This makes a universal pan-cancer test difficult to set up.

Thus, there is a strong need for a new method capable of assessing the potential of DNA methylation, in particular circulating DNA methylation, as a universal tumor biomarker, and to develop new highly sensitive strategies to detect cancer-specific signatures, preferably in a blood sample.

### SUMMARY OF THE INVENTION

The invention concerns a method for determining a CpG methylation profile of at least one DNA sequence of interest or any fragment thereof, wherein the method comprises:
a) clustering a set of sub-sequences obtained from a DNA sequence of interest into clusters of sub-sequences;
b) selecting, for and from each cluster, one sub-sequence as a reference sequence among the sub-sequences of the cluster,
c) aligning the reference sequences of said clusters by allowing the alignment on positions of CpG dinucleotides,
d) for each cluster, aligning the remaining sub-sequences on selected reference sequences; and
e) determining the CpG methylation status of each sub-sequence by determining at each CpG site of the sub-sequence if the CpG dinucleotide is methylated or not, thereby determining a CpG methylation profile comprising a CpG methylation level and/or a proportion of CpG methylation haplotype of the sub-sequences.
wherein the DNA sequence of interest is or comprises a repeated sequence, said repeated sequence being distributed throughout the subject's genome, and preferably comprising high density of CpG dinucleotides;
wherein, the method optionally comprises a first step of obtaining or providing a set of sub-sequences of said DNA sequence of interest, and
wherein the method optionally comprises repeating some, or each, of steps a) through e) with other sets of sub-sequences from the DNA sequence of interest.

In particular, the repeated sequence is a retrotransposon such as LINE, HERV, SINE, SVA, or a subfamily thereof such as in particular LINE-1, L1PA, HERV-K and Alu, or a satellite repeat such as Sat2 or Sat3 element, preferably a LINE-1 retrotransposon or any fragment or variant thereof, even more preferably a LINE-1 retrotransposon such as described under SEQ ID NO: 2 or 29 or any fragment or variant thereof.

The invention also concerns a computer-implemented method of training a classifier for accurately distinguishing between a healthy CpG methylation profile and a cancerous CpG methylation profile, said method comprising:
a) providing a training set of CpG methylation profiles of DNA sequences of interest or sub-sequences thereof, said DNA sequences of interest being repeated and distributed throughout a genome and comprising high density of CpG dinucleotides, or preprocessed information obtained from said training set of CpG methylation profiles of DNA sequences of interest or sub-sequences thereof, as an input to a classifier, said training set of CpG methylation profiles comprising CpG methylation profiles of DNA sequences, or of sub-sequences thereof, from subjects identified as healthy subjects and from subjects identified as cancerous subjects; and,
b) generating an output of the classifier for each CpG methylation profile input of DNA sequence of interest or sub-sequences thereof, said output classifying the CpG methylation profile input of DNA sequence of interest or sub-sequences thereof as a healthy CpG methylation profile or as a cancerous CpG methylation profile;
wherein the CpG methylation profile comprises a CpG methylation level and/or proportion of CpG methylation haplotypes of the DNA sequence or sub-sequences thereof.

In particular, the CpG methylation profiles of the DNA sequences of interest or sub-sequences thereof are determined by the method described herein.

The invention also concerns an in vitro or in silico method of determining the health status of a subject, in particular of determining if the subject is a healthy subject or a subject suffering from cancer or cancer relapse, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
(b) using the classifier to identify the CpG methylation profile of the DNA sequence of interest or sub-sequences thereof of said subject as a healthy CpG methylation profile or as a cancerous CpG methylation profile as an output of the classifier.

The invention also concerns an in vitro or in silico method of determining the origin of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile from different tumors origins, and
(b) using the classifier to identify the CpG methylation profile of the DNA sequence of interest or sub-sequences thereof of said subject as a healthy CpG methylation profile or as a cancerous CpG methylation profile from a particular tumor origin to determine the origin of the tumor from the subject as an output of the classifier.

The invention also concerns an in vitro or in silico method of determining the stage of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile of different stages, and
(b) using the classifier to identify the CpG methylation profile of the DNA sequence of interest or sub-sequences thereof of said subject as a healthy CpG methylation profile or as a cancerous CpG methylation profile of a particular stage to determine the stage of the tumor from the subject as an output of the classifier.

The invention also concerns an in vitro or in silico method of monitoring the response to an anti-cancer treatment of a subject suffering from cancer, wherein the method comprises:
(a) providing at least one DNA sequence of interest or sub-sequences thereof from a first liquid biopsy from a subject suffering from cancer before the administration of the anti-cancer treatment to the subject as a first input, said DNA sequence of interest being repeated through the subject genome and comprising high density of CpG sites or a fragment thereof, or preprocessed information obtained from said first liquid biopsy, and a second liquid biopsy comprising at least one DNA sequence of interest or sub-sequences thereof from said subject after the administration of an anti-cancer treatment as a second input, or preprocessed information obtained from said second liquid biopsy, to a classifier trained to distinguish between DNA sequence having a healthy CpG methylation profile and DNA sequence having a cancerous CpG methylation profile; and
(b) using the classifier to identify each CpG methylation profile of each DNA sequence of the first liquid biopsy as having a healthy CpG methylation profile or a cancerous CpG methylation profile as a first output of the classifier, and to identify each CpG methylation profile of each DNA sequence of the second liquid biopsy as having a healthy CpG methylation profile or a cancerous CpG methylation profile as a second output of the classifier, and
wherein a number of DNA sequence of interest classified as having a healthy CpG methylation profile in the second output of the classifier which is above a number of DNA sequence of interest classified as having a healthy CpG methylation profile in the first output of the classifier is indicative that the subject is responsive to said anti-cancer treatment, and wherein a number of DNA sequence of interest classified as having a healthy CpG methylation profile in the second output of the classifier which is equal to or below a number of DNA sequence of interest classified as having a healthy CpG methylation profile in the first output of the classifier is indicative that the subject does not respond to said anti-cancer treatment.

The invention also concerns an in vitro or in silico method of assessing the potency of a compound to revert a cancerous CpG methylation profile of a DNA sequence of interest from a subject suffering from cancer into a healthy CpG methylation profile, wherein the method comprises:
(a) providing a DNA sequence of interest or sub-sequences thereof from the subject having been treated with a compound, said DNA sequence of interest being repeated and distributed throughout the subject's genome and comprising high density of CpG dinucleotides or any fragment thereof, or preprocessed information obtained from said at least one DNA sequence of interest or sub-sequences thereof, as an input to a classifier trained to distinguish between DNA sequence having a healthy CpG methylation profile or a cancerous CpG methylation profile; and
(b) using the classifier to detect DNA sequences having a healthy CpG methylation profile and/or DNA sequences having a cancerous CpG methylation profile as an output of the classifier, wherein an amount of DNA sequences having a healthy methylation profile above a reference amount of DNA sequences having a healthy methylation profile obtained from the subject before any treatment with the compound is indicative that the compound is able to revert the cancerous CpG methylation profile into a healthy CpG methylation profile.

The invention also concerns an in vitro or in silico method of predicting the ability of a compound to treat a cancer comprising assessing the potency of a compound to revert a cancerous CpG methylation profile of a DNA sequence of interest from a subject into a healthy CpG methylation profile,
wherein an amount of DNA sequences classified as having a healthy CpG methylation profile, which is above the reference amount is indicative that said compound is useful in the treatment of said cancer.

In particular, in the test method disclosed herein, the CpG methylation profiles of the DNA sequence of interest or sub-sequence thereof is determined by the method of determination of CpG methylation profiles as disclosed herein.

In some embodiments, the classifier is trained according to the training methods disclosed herein.

In some embodiments, the DNA sequence of interest is a circulating cell-free DNA (cfDNA) sequence.

The invention also concerns a computing system comprising:
a memory storing at least one instruction of a classifier trained according to the training method disclosed herein, and
a processor accessing to the memory for reading the aforesaid instruction(s) and executing any of the method according to the invention.

The invention also concerns a kit of primers or probes targeting sub-sequences of a DNA sequence encoding a LINE-1 retrotransposon, preferably a LINE-1 retrotransposon such as described under SEQ ID NO: 2 or 29, said kit comprising at least 4 primers or probes selected from the group of primers or probes having a sequence as set forth in SEQ ID NO: 11, 12, 13, 14, 15, 16, 17, 18, 19, 20, 21, 22, 23, 24, 25 or 26 respectively or a sequence having at least 85% identity thereto.

The invention also concerns the use of the kit for amplifying sub-sequences of a DNA sequence encoding a LINE-1 retrotransposon, preferably a LINE-1 retrotransposon such as described under SEQ ID NO: 2 or 29, for the diagnosis of cancer, such cancer being preferably selected from the group consisting of colon cancer, breast cancer, lung cancer, uveal melanoma cancer, ovary cancer and stomach cancer.

### DETAILED DESCRIPTION OF THE INVENTION

There is a need for improved methods of detecting multiple types of cancer. This includes the need for efficient cancer screening at an early stage.

Remarkably, cancer-related hypomethylation has been reported in almost all classes of repeated sequences, from dispersed retrotransposons to clustered satellite repeated DNA, and in multiple forms of cancers.

To obtain a global representation of the hypomethylation occurring during carcinogenesis and to increase sensitivity, the inventor chose to target retrotransposons in particular, such as the Long-Interspersed Element-1 family (L1). Indeed, these elements have thousands of copies per genome and are hypomethylated in multiple cancers.

Two studies have explored L1 global methylation profiles from plasma of lung and colorectal cancers, using qPCR-based methods, but reported low detection sensitivity, below 70%. Indeed, detecting methylation profiles at the single base-pair resolution at repeats requires sophisticated downstream analysis because these sequences are inherently difficult to map. To overcome this, the inventors have developed computational tools to accurately align sequencing data without a reference genome. The inventors have implemented prediction models, trained by machine learning algorithms, integrating patterns of methylation, globally and at the single molecule level.

The present invention dramatically increases the sensitivity of DNA detection in a cost-effective manner, providing an optimal trade-off between the number of targeted regions and sequencing depth.

The description also relates in particular to a new method that uses multi-cancer hypomethylation markers in order to capture a wide range of tumor alleles and covers the heterogeneous profiles of cancer patients in a single test. The method interrogates selected regions, which provide genome-wide information because repetitive elements, such as retrotransposons, hold half of the CpG sites present in the human genome. This allows to generate methylation profiles from minute amounts of DNA, down to a few nanograms, with high precision and high coverage using affordable sequencing depth. This method is widely usable for the development of routine clinical tests.

The greatest originality and competitive advantage of this invention is to interrogate DNA methylation at the level of repeated sequences. Hypomethylation of repeats being common to many, if not all, cancer types, it is an interesting marker for pan-cancer detection. Previous studies have left these regions aside because they are inherently difficult to map and differentially methylated regions (DMR) analysis is commonly performed on mapped data. The inventors have developed a new solution to detect methylation profiles at repeats with a single base-pair resolution, without resorting to mapping on a reference genome. This allows to retain most of the data, which is essential for achieving high sensitivity as well as working with minute amounts of DNA. As herein shown, the method herein disclosed by inventors has demonstrated high performance in the detection of cancer samples and was assessed in six different cancer types, including three at early stages, showing its interest in pan-cancer diagnosis.

### Definitions

In order that the present invention may be more readily understood, certain terms are defined herein. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, “CpG” or “CG” is used interchangeably and refers to cytosine (“C”) and guanine (“G”) nucleotides that are connected by a phosphodiester bond and particularly refers to specific CG dinucleotides located in a “CpG site”. In mammals, DNA methylation occurs mostly at CpG dinucleotides.

In a particular aspect, cytosine residues of CpG dinucleotides are methylated to 5-methylcytosine.

As used herein, “CpG island” refers to stretches of DNA, in particular circulating cell-free DNA (cfDNA, also herein identified as “cell-free DNA”), where the frequency of CpG sites is greater relative to other regions of the DNA. To be recognized as a CpG island, a sequence must satisfy the following criteria: (G+C) content of 0.50 or greater; a CpG dinucleotide ratio of 0.60 or greater; and both occurring within a sequence window of 200 bp or 500 bp.

As used herein, the term “methylation” refers to methylation of cytosine residues, in particular to methylation of C5 position of cytosine and/or N4 position of cytosine, preferably of methylation of C5 position of cytosine. A cytosine comprised in a CpG site that can be methylated is referred to as a “cytosine susceptible to be methylated.” A cytosine that is methylated is referred to as a “methylated cytosine”. In some instances, methylation specifically refers to methylation of cytosine residues present in CpG sites.

As used herein, the term “differentially methylated” describes a CpG methylation site for which the methylation profile differs between a first condition and a second condition, e.g., a healthy versus cancerous condition.

As used herein, the term “hypomethylation” refers to lower levels of methylation that can be reported at the level of a CG dinucleotide, of a nucleic region or of a CpG island in a state of interest as compared to a reference state (e.g., at least one less methylated cytosine in a cancer condition than in a healthy control).

As used herein, the term “hypermethylation” refers to a higher level of methylation that can be reported at the level of a CG dinucleotide, of a nucleic region or of a CpG island in a state of interest as compared to a reference state (e.g., at least one more methylated cytosine in a cancer condition than in a healthy control).

As used herein, the term “sub-sequences of a DNA sequence” refers to a part or fragment of an original DNA sequence. A subsequence particularly consists of a consecutive run of nucleic acids from the original DNA sequence. A subsequence of a DNA sequence is shorter (i.e., comprises less nucleic acids) than the original DNA sequence.

As used herein, the term “amplicon” or “amplicon molecule” refers to a nucleic acid molecule generated by amplification of a template nucleic acid molecule, such as a cfDNA, or a nucleic acid molecule having a sequence complementary thereto, or a double-stranded nucleic acid including any such nucleic acid molecule. As used herein, the term “oligonucleotide primer”, or “primer”, refers to a nucleic acid molecule used, capable of being used, or for use in, generating amplicons from a template nucleic acid molecule.

Under amplification-permissive conditions (e.g., in the presence of nucleotides and a DNA polymerase, and at a suitable temperature and pH), an oligonucleotide primer can provide a point of initiation of amplification from a template to which the oligonucleotide primer hybridizes. Typically, an oligonucleotide primer is a single-stranded nucleic acid between 5 and 200 nucleotides in length. Those of ordinary skill in the art will appreciate that optimal primer length for generating amplicons from a template nucleic acid molecule can vary with conditions including temperature parameters, primer composition, and transcription or amplification method. A pair of oligonucleotide primers, as used herein, refers to a set of two oligonucleotide primers that are respectively complementary to a first strand and a second strand of a template double-stranded nucleic acid molecule. First and second members of a pair of oligonucleotide primers may be referred to as a “forward” oligonucleotide primer and a “reverse” oligonucleotide primer, respectively, with respect to a template nucleic acid strand, in that the forward oligonucleotide primer is capable of hybridizing with a nucleic acid strand complementary to the template nucleic acid strand, the reverse oligonucleotide primer is capable of hybridizing with the template nucleic acid strand, and the position of the forward oligonucleotide primer with respect to the template nucleic acid strand is 5′ of the position of the reverse oligonucleotide primer sequence with respect to the template nucleic acid strand. It will be understood by those of ordinary skill in the art that the identification of a first and second oligonucleotide primer as forward and reverse oligonucleotide primers, respectively, is arbitrary in as much as these identifiers depend upon whether a given nucleic acid strand or its complement is utilized as a template nucleic acid molecule.

As used herein, the term “probe” refers to a single- or double-stranded nucleic acid molecule that is capable of hybridizing with a complementary target, such as DNA, a cfDNA or an amplicon, and includes a detectable moiety. In some instances, e.g., as set forth herein, a probe is a capture probe useful in the detection, identification and/or isolation of a target sequence, such as a gene sequence.

As used herein, the “sequence identity” between two sequences is described by the parameter “sequence identity”, “sequence similarity” or “sequence homology”. In the context of the present invention, the “sequence identity” between two sequences (A) and (B) is determined by comparing two sequences aligned in an optimal manner, through a window of comparison. Said sequences alignment can be carried out by methods well-known in the art, for example, using the Needleman-Wunsch global alignment algorithm, or the Smith-Waterman local alignment algorithm. The analysis software matches similar sequences using similarity measures attributed to various deletions and other modifications. Once the total alignment has been obtained, the identity percentage can be obtained by dividing the total number of identical nucleic acid residues aligned by the total number of nucleic acid residues contained in the longest sequence between the sequences (A) and (B). To compare two nucleic acid sequences, one can use, for example, the BLAST or EMBOSS Needle tool. EMBOSS Needle creates an optimal global alignment of two sequences using the Needleman-Wunsch algorithm.

As used herein, the term “diagnosis” refers to determining whether, and/or the qualitative of quantitative probability/likelihood that, a subject has or will develop a disease, disorder, condition, or state. For example, in diagnosing a cancer, the diagnosis can include a determination of the risk, type, stage, malignancy, or other classification of a cancer. In some instances, e.g., as sort forth herein, a diagnosis can be, or include, determining the prognosis and/or likely response to one or more general or particular therapeutic agents or regimens.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain aspects, such a term refers to the improvement or eradication of a disease, a disorder, an infection or symptoms associated with it. In other aspects, this term refers to minimizing the spread or the worsening of cancer. Treatments according to the present invention do not necessarily imply 100% or complete treatment. Rather, there are varying degrees of treatment recognized by one of ordinary skill in the art as having a potential benefit or therapeutic effect. Preferably, the term “treatment” refers to the application or administration of a composition including one or more active agents to a subject who has a disorder/disease.

As used herein, the term “classifier performance” refers to the predictive capabilities of machine learning models. Different types of classification performance metrics are used to measure the performance of a classifier, such as accuracy, sensitivity, specificity or area under the ROC curve.

As used herein, the term “computer-implemented method” refers to a method which involves a programmable apparatus/device, in particular a computer, computer network, or readable medium carrying a computer program, in which at least one step of the method is performed by using at least one computer program. A computer-implemented method may further comprise at least one step that is not performed by using a computer program.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The articles “a” and “an” are used herein to refer to one or to more than one (i.e., to at least one) of the grammatical object of the article. By way of example, “an element” refers to one element or more than one element.

The term “about”, when used herein in reference to a value, refers to a value that is similar, in context, to the referenced value. In general, one of ordinary skill in the art, familiar with the context, will appreciate the relevant degree of variance encompassed by the term “about” in that context. For example, in some embodiments, e.g., as set forth herein, the term “about” can encompass a range of values within 25%, 20%, 19%, 18%, 17%, 16%, 15%, 14%, 13%, 12%, 11%, 10%, 9%, 8%, 7%, 6%, 5%, 4%, 3%, 2%, 1%, or within a fraction of a percent, of the referred value.

### Methods According to the Invention

### Determination of CpG Methylation Profile

In a first aspect, the invention concerns a method, in particular a computer implemented method, for determining a CpG methylation profile of a DNA sequence of interest, wherein the method comprises:
a) clustering a set of sub-sequences obtained from a DNA sequence of interest into clusters of sub-sequences;
b) selecting, for and from each cluster, one sub-sequence as a reference sequence among the sub-sequences of the cluster,
c) aligning the reference sequences of said clusters by allowing the alignment on positions of CpG dinucleotides,
d) for each cluster, aligning the remaining sub-sequences on selected reference sequences; and
e) determining the CpG methylation status of each sub-sequence by determining at each CpG site of the sub-sequence if the CpG dinucleotide is methylated or not, thereby determining a CpG methylation profile comprising a CpG methylation level and/or a proportion of CpG methylation haplotype of the sub-sequences.
wherein the DNA sequence of interest is a DNA sequence from a subject encoding a repeated sequence distributed throughout the subject's genome, and preferably comprising high density of CpG dinucleotides, or any fragment of said repeated sequence;
wherein, the method optionally comprises a first step of obtaining or providing a set of sub-sequences of said DNA sequence of interest, and
wherein the method optionally comprises performing/repeating (one or several times) some, or each, of steps a) through e) with other sets of sub-sequences.

The method may also comprise an additional final step of comparing the CpG methylation profiles (as determined in step e)) to each other in order to optimize the CpG methylation profile(s).

In a particular embodiment, the DNA is obtained/isolated/extracted from, or comprised/included in, a biological sample, in particular a biological sample from a subject. Such biological sample is described in particular below under the paragraph “Subject and biological sample”.

Preferably, the DNA sequence of interest is retrieved from cfDNA. Cellular DNA methylation patterns are conserved in cell-free DNA (cfDNA).

As used herein, “circulating cell-free DNA” and “cfDNA” refer to DNA fragments released from cells to body fluid such as blood plasma. This term includes normal circulating cell free DNA, circulating tumor DNA (ctDNA), cell-free mitochondrial DNA (cf-mtDNA), and cell-free fetal DNA (cf-fDNA). The terms “Circulating tumor DNA” and “ctDNA” are used interchangeably and refer to a portion of circulating cell-free DNA, which is released from cancer cells.

Preferably, the sub-sequence of the DNA of interest is a sequence comprising a high density of CpG sites.

As used herein “high density of CpG dinucleotides” or “high CpG dinucleotide density” refer to the density of CpG dinucleotides normalized by the densities of G and C nucleotides in a DNA sequence, or refer to the number of CpG dinucleotides in a DNA sequence. A density of CpG dinucleotides is considered “high” when a ratio of observed to expected CpG dinucleotides (CpG observed/CpG expected) is of 0.6 or greater.

In one embodiment, the DNA of interest comprises at least 10, 15, 20, 25, 30, 35, 40, 45 or 50 CpG sites. Particularly, the DNA of interest comprises between 20 and 40 CpG sites, more particularly between 30 and 35 CpG sites. Such CpG sites are particularly distributed among one or more DNA subsequence(s).

In a particular embodiment, the DNA sequence of interest is a DNA sequence comprising differentially methylated CpG dinucleotides. In particular, such DNA sequence is known to be heavily methylated in healthy subjects and hypomethylated in subjects suffering from cancer.

In a particular embodiment, the DNA sequence and/or subsequence of interest comprises at least one differentially methylated region, preferably comprising high density of CpG sites.

As used herein, the term “differentially methylated region” or “DMR” refer to a DNA sequence that includes one or more differentially methylated CpG sites between a first and second condition, e.g., a healthy versus cancerous condition. A DMR that includes a greater or higher number or frequency of methylated CpG sites in a selected condition of interest, such as a cancerous state, can be referred to as a “hypermethylation DMR”. A DMR that includes a lower number or frequency of methylated sites in a selected condition of interest, such as a cancerous state, can be referred to as a “hypomethylation DMR”. The term “DMR” also designates DNA sequences that have a different methylation profile, i.e., a different methylation level and/or methylation pattern, between a first and second condition, e.g., healthy versus cancerous condition. In a particular aspect herein described, the DMR is a subsequence of a DNA sequence of interest. In a particular aspect herein described, the DMR is an amplicon, for example produced by amplification using oligonucleotide primers, e.g., a pair of oligonucleotide primers selected for the amplification of the DMR or for the amplification of a DNA subsequence of interest. In a particular aspect herein described, the DMR is a DNA region amplified by a pair of oligonucleotide primers, for example the region having the sequence of, or a sequence complementary to, an oligonucleotide primer. Preferably, the DMR has a high density of CpG sites.

The DNA envisioned by the invention is a DNA sequence, preferably a cfDNA, that comprises a repeated sequence or any fragment thereof. Preferably, such repeated sequence comprises one or more DMR.

“Repetitive sequence”, “repeated sequences” or “repeats” are used interchangeably herein and refer to multiple copies of nucleotide sequences in the genome. They are abundantly distributed in the genomes of eucaryotes. Two large families of repetitive sequences can be readily recognized, “tandem repeats” and “dispersed repeats”.

Preferably, the repeated sequence is present in at least 100, at least 1 000, at least 10 000, at least 100 000 or at least 1 000 000 copies in the subject's genome.

In a particular aspect, the repeated sequence is selected from the group consisting of LINE, HERV, SINE, SVA, Sat2 and Sat3 elements, including their subfamilies such as L1PA, HERV-K and Alu, or any variant (i.e., similar version) thereof. The variant sequence of a particular (reference) sequence is a sequence having at least 80%, 85%, 90%, 95%, 96%, 97%, 98% or 98% sequence identity with said particular (reference) sequence.

In an embodiment, the repeated sequence according to the invention is a tandem repeat sequence. Tandem repeats are composed by one or more nucleotides repeated in a block or an array in a head-to-tail orientation and are usually non-coding sequences. According to the size of the repeated unit and the total length, they can be further classified in satellites (sat1, sat2, sat3, centromeric alpha-satellites, telomeres), minisatellites (variable number of tandem repeats (VNTRs)) and microsatellites (simple sequence repeats, SSRs).

Preferably, the tandem repeated sequence is a Sat2 or Sat3 satellite, in particular a Human Satellites 2 or 3, or any fragment or variant thereof. Sat2 and Sat3 satellites are particularly described in Altermose et al. PLOS Computational Biology 2014 Volume 10 Issue 5 e1003628. SAT2/3 are enriched for tandem repeats of the pentamer GGAAT, as well as diverged sequences including CGGAT.

In another embodiment, the repeated sequence is a dispersed repeat, also called “interspersed repeat”. Transposable elements (transposons), such as DNA transposons and retrotransposons, are interspersed repeats.

In a particular embodiment, the repeated sequence is a transposon or a retrotransposon. Transposable elements or transposons are small DNA segments capable of replicating and inserting copies of DNA at random sites in the same or a different chromosome. In eukaryotes such as humans, transposons may be classified as Class I or Class II. In particular, class I elements (so-called copy-and-paste retrotransposons) use reverse transcribed RNA intermediates to produce copies of themselves, and class II elements (so-called cut-and-paste DNA transposons) excise from a donor site to reintegrate elsewhere in the genome (Wicker, T. et al. Nat. Rev. Genet. 8, 973-982 (2007). Thus, the repeated sequence can be a transposon of class I or II.

Preferably, the repeated sequence is a class I transposon, i.e., a retrotransposon. In particular, the repeated sequence is an evolutionarily young retrotransposon, preferably specific to human or primate genome.

In an embodiment, the repeated sequence is a SINE or SINE-VNTR-Alu (SVA) element or any fragment or variant thereof.

The term “Short Interspersed Nuclear Element” (SINE) refers to retrotransposons that constitute one of the main components of the genomic repetitive fraction. More than one million copies of short interspersed elements (SINEs), a class of retrotransposons, are present in the mammalian genomes, particularly within gene-rich genomic regions.

The term “SINE-VNTR-Alu” (SVA) refers herein to non-autonomous hominid specific retrotransposons that are known to be associated with disease in humans. SVAs are evolutionarily young and presumably mobilized by the LINE-1 reverse transcriptase in trans. SVAs elements impact the host through a variety of mechanisms including insertional mutagenesis, exon shuffling, alternative splicing, and the generation of differentially methylated regions (DMR). A canonical SVA is on average about 2 kilobases (kb) but SVA insertions may range in size from 700-4 000 base pairs (bp). SVA retrotransposons are particularly described in Hanks and Kazazian (Semin Cancer Biol. 2010 August; 20(4): 234-245) and Gianfrancesco et al. Neuropeptides. 2017 August; 64: 3-7).

In an embodiment, the repeated sequence is a Alu element or any fragment or variant thereof.

The term “Alu transposon” or “Alu element” refers to a short stretch of DNA originally characterized by the action of the Arthrobacter luteus (Alu) restriction endonuclease. Alu elements are the most abundant transposable elements, containing over one million copies dispersed throughout the human genome. Alu elements are about 300 base pairs long and are therefore classified as short interspersed nuclear elements (SINEs) among the class of repetitive DNA elements. The typical structure of a Alu element is 5′—Part A—A5TACA6—Part B—PolyA Tail—3, where Part A and Part B are similar nucleotide sequences.

In a preferred embodiment, the Alu retrotransposon has a nucleotide sequence such as described under SEQ ID NO: 1 or a similar sequence, i.e., a sequence having at least 80%, 85%, 90%, 95%, 96%, 97%, 98% or 98% sequence identity thereto.

In an embodiment, the repeated sequence is a HERV element or any fragment or variant thereof. Preferably, the repeated sequence is an HERV-K element, which belong to a subfamily of HERV elements. The terms “Human endogenous retrovirus K” (HERV-K) or “Human teratocarcinoma-derived virus” (HDTV) or “Human mouse mammary tumor virus like-2” (HML-2) are used interchangeably and refer to a family of human endogenous retroviruses. Expression of HERV-K in humans has been associated with various types of cancer. Human genome contains hundreds of copies of HERV-K. HERV-K elements are particularly described in Agoni et al. (Front Oncol. 2013; 3: 180) and in Garcia-Montojo M et al. (Crit Rev Microbiol. 2018 November; 44(6):715-738).

In another embodiment, the repeated sequence is a LINE element, preferably a LINE-1 retrotransposon or any fragment or variant thereof. Preferably, the repeated sequence is a primate-specific copy (for example L1PA or L1HS) of the LINE-1 retrotransposon.

The terms “LINE-1”, “LINE1” or “L1” are used interchangeably herein and refer to reverse transcription transposon LINE-1 (also known as Long Spreading Element-1 or Long Distribution Element-1). LINE1 are class I transposable elements and belong to the group of long interspersed nuclear elements (LINEs). LINE-1 retrotransposon comprises approximately 17% of the human genome. A typical LINE-1 element is approximately 6,000 base pairs (bp) long and consists of two non-overlapping open reading frames (ORF) which are flanked by untranslated regions (UTR) and target site duplications.

In an embodiment, the LINE-1 retrotransposon has a nucleotide sequence such as described under SEQ ID NO: 2 or SEQ ID NO: 29 or a similar sequence, i.e., a sequence having at least 80%, 85%, 90%, 95%, 96%, 97%, 98% or 98% sequence identity thereto.

In a preferred embodiment, the LINE-1 retrotransposon has a nucleotide sequence such as described under SEQ ID NO: 29 or a similar sequence, i.e., a sequence having at least 80%, 85%, 90%, 95%, 96%, 97%, 98% or 98% sequence identity thereto.

In an embodiment, the DNA repeated sequence of interest comprises at least 1, 2, 3, 4, 5, 6, 7, 8, 9, 10, 20, 30, 60 or 90 CpG dinucleotides, preferably at least 30 or at least 90 CpG dinucleotides.

The method according to the invention may comprise a step of pretreatment of the DNA or sub-sequence thereof.

In an embodiment, the DNA sequence of interest, for example the cfDNA of interest, is treated to deaminate non-methylated cytosine(s) prior to the determination of the CpG methylation profile of said DNA sequence of interest. Such treatment of DNA can be used to deaminate unmethylated cytosine to produce uracil in DNA. Upon sequencing and/or amplification using a specific probe and/or primers, the methylation status of the DNA can thus be detected based on identification of a change in base from cytosine to uracil or thymine.

The deamination of cytosines may be performed by any method known in the art such as a treatment using anyone, or any combination, of bisulfite reagents such as sodium bisulfite or enzyme(s) such as Tet methylcytosine dioxygenase 2 (TET2), T4-phage beta-glucosyltransferase (T4-BGT) and Apolipoprotein B mRNA editing enzyme catalytic subunit 3A (APOBEC3A) enzymes, for example such as described in Vaisvila et al., Genome Res. 2021. 31: 1280-1289.

Other methods to analyse methylation status do not necessarily use cytosine deamination (such as TET-assisted pyridine borane sequencing (TAPS) and Immunoprecipitation-based method coupled to deep sequencing (MeDIP)).

For example, it is possible to use DNA-methylation-sensitive or DNA-methylation-specific restriction enzymes which distinguish molecules which are methylated or not. It is also possible to use quantitative PCR (qPCR), droplet digital PCR (ddPCR) methods that target methylated versus unmethylated molecules. Another method to distinguish the methylation status of cytosines without conversion is the direct sequencing using the Oxford Nanopore Technologies (ONT) which accurately detects 5mC changes even from plasma DNA (Cheng et al., Clin. Chem. 61, 1305-1306 (2015)).

Preferably, the DNA sequence of interest is treated with a bisulfite reagent, preferably with sodium bisulfite.

Bisulfite reagents usable in the context of the present invention, include, for example and among others, bisulfite, disulfite, hydrogen sulfite, or any combination thereof, which reagents can be useful in distinguishing methylated and unmethylated nucleic acids. Bisulfite interacts differently with cytosine and 5-methylcytosine. In typical bisulfite-based methods, contacting of DNA with bisulfite deaminates unmethylated cytosine to uracil, while methylated cytosine remains unaffected, and methylated cytosines, but not unmethylated cytosines, are selectively retained. The same applies for EM-seq (deamination of unmethylated cytosines with enzymes). Thus, uracil or thymine residues stand in place of, and provide an identifying signal for, unmethylated cytosine residues, while remaining (methylated) cytosine residues provide an identifying signal for methylated cytosine residues. Processed samples can be analyzed, e.g., by next generation sequencing (NGS) or targeted bisulfite NGS/deep sequencing. In particular, when uracil (U) is copied as thymine (T) during an amplification step (such as PCR amplification) changes in base from cytosine to thymine are identified.

Various methylation assay procedures can be used in conjunction with a bisulfite treatment to determine methylation profiles of a DNA sequence of interest. Such assays can include, for example and among others, the sequencing of bisulfite-treated nucleic acid, PCR (e.g., with sequence-specific amplification), Methylation-Sensitive High Resolution Melting (MS-HRM) PCR (see, e.g., Hussmann 2018 Methods Mol Biol. 1708:551-571), Quantitative Multiplex Methylation-Specific PCR (QM-MSP) (see, e.g., Fackler 2018 Methods Mol Biol. 1708:473-496), Methylation Specific Nuclease-assisted Minor-allele Enrichment (MS-NaME) (see, e.g., Liu 2017 Nucleic Acids Res. 45(6):e39), pyrosequencing and Methylation-sensitive Single Nucleotide Primer Extension (Ms-SNuPE™) (see, e.g., Gonzalgo 2007 Nat Protoc. 2(8):1931-6).

In an embodiment, the DNA sequence is treated by TET-assisted pyridine borane sequencing (TAPS). TAPS specifically transforms only the methylated cytosines and preserves DNA integrity, allowing very little DNA to be analyzed. This also improves the downstream analysis, as the resulting reads preserve their full complexity. In a particular embodiment, sub-sequence(s) of a DNA of interest is/are amplified from a bisulfite-treated DNA sample. In a particular embodiment, high-throughput and/or next-generation sequencing techniques is/are used to achieve base-pair-level/scale resolution of a DNA sequence, allowing analysis of its methylation profile.

In an embodiment, the DNA sequence of interest is not treated to deaminate non-methylated cytosines prior to the determination of the CpG methylation profile. The man skilled in the art is aware of techniques that allows to identify CpG methylation without the need of deamination treatments. Single molecule real-time (SMRT) sequencing theoretically offers the opportunity to directly assess certain base modifications of native DNA molecules without any prior chemical/enzymatic conversions and PCR amplification, using kinetic signals of a DNA polymerase. For example, In Nanopore sequencing devices (e.g., by Oxford Nanopore Technologies) and single molecule real-time (SMRT) sequencing (e.g., by Pacific BioSciences, PacBio), electrolytic current signals are sensitive to base modifications, such as 5-methylcytosine (5-mC) and allows the detection of native CpG methylation sites (Cheng et al., Clin. Chem. 61, 1305-1306 (2015)).

In an embodiment, the method according to the invention comprises a step of amplification of the DNA sequence of interest or subsequence(s) thereof. In particular, the DNA sequence or DNA subsequence is amplified, for example by polymerase chain reaction (PCR), preferably multiplex PCR, before clustering.

As used herein, the term “amplification” refers to the use of a template nucleic acid molecule, such as a cfDNA, in combination with various reagents to generate additional nucleic acid molecules from the template nucleic acid molecule, e.g. “amplicons”, which additional nucleic acid molecules may be identical to or similar to (e.g., at least 80% identical, e.g., at least 85%, 90%, 91%, 92%, 93%, 94%, 95%, 96%, 97%, 98%, 99%, or 100% identical to) the template nucleic acid molecule, a sequence complementary thereto, and/or a segment thereof.

The process of DNA amplification can be performed by any methods known by the man of ordinary skill in the art, such as polymerase chain reaction (PCR). Preferably, the method for determining a CpG methylation profile is a PCR-based method.

Thus, in an embodiment, the sub-sequence of the DNA of interest is an amplicon.

The DNA subsequence may be amplified using specific primer pairs complementary to the DNA sequence of interest, such as LINE-1 for example. The man skilled in the art knows how to design suitable pairs of primers, for example using alignment tools.

In an embodiment, the amplicons are selected from the group consisting of amplicon #1, #2, #3, #4, #5, #6, #7 or #8, and any combination thereof, for example such as describes in FIG. 1 herein and/or under the SEQ ID NO: 3, 4, 5, 6, 7, 8, 9 and 10, respectively. In particular, the method comprises the study of amplicons #1, #2, #4 #5 #6 #7 and #8, and optionally of amplicon #3.

Preferably, the amplicons studied in the method according to the invention comprise, or consist essentially of, amplicons having a sequence selected from the group consisting of SEQ ID NO: 3, 4, 5, 6, 7, 8, 9, 10, and any sequence having at least 85, 90, 95, 98, or 99% sequence identity thereto.

Alternatively, the DNA subsequence may be amplified with universal or degenerated primers.

The primers used to amplify the DNA subsequence may comprise adapter(s) such as Unique Molecular Identifier(s) (UMI(s)) or a unique dual index(es) (UDI(s)).

UMIs may be of any suitable length to produce a sufficiently large number of unique UMIs. In a particular aspect, a UMI may be between 5 and 20 nucleotides in length. Therefore, each UMI may be approximately 5, 6, 7, 8, 9, 10, 11, 12, 13, 14, 15, 16, 17, 18, 19 or 20 nucleotides in length. In one embodiment, a UMI is a nucleotide sequence of 16 nucleotides in length.

In an embodiment, primers are methylation-independent with 0 to 2 CpG sites included and preferably none CpG site toward the 5′ end of the primers.

In an embodiment, the DNA sequence targeted by the primers ranged from 100 to 200 bp, preferably from 101 bp to 150 bp.

In one embodiment, the method further comprises a step of eliminating PCR replicates after PCR amplification of the DNA sequence or sub-sequence of interest. A common practice to eliminate PCR duplicates is to remove all but one read of identical sequences, assuming that such reads have been created from the same DNA molecule by PCR. Preferably, the methods according to the invention relies on the use of primers comprising Unique molecular identifiers (UMIs) to accurately detect PCR duplicates. The primer may further contain common or universal sequence(s) CS1 and/or CS2. The common sequence can for example consists of, or comprises, common sequence 1 (CS1) (5′-ACACTGACGACATGGTTCTACA-3′ SEQ ID NO: 27) and/or common sequence 2 (CS2) (5′-TACGGTAGCAGAGACTTGGTCT-3′ SEQ ID NO: 28) universal primer sequence(s).

In an embodiment, the method according to the invention preferably comprises a step of capturing the DNA sequence(s), preferably the cfDNA sequence(s). In particular, the DNA sequence or DNA subsequence is captured before amplification.

In a particular embodiment, one or more probes are used to capture DNA sequence(s) or subsequence(s) of interest.

In an embodiment, the method comprises using at least 20, 50, 100, 200, 250 or 300 probes targeting different regions of a DNA sequence of interest, in particular between 200 and 250, preferably between 210 and 230 different probes.

In a very particular embodiment, the method comprises using probes of between 100 and 150 bp, preferably 120 bp, which start every 20 to 30 bp, preferably every 24 bp.

In another embodiment, the method comprises using 4, 5, 6, 7, 8, 9, 10, 11, 12, 13, 14, 15, 16, 17, 18, 19 or 20 probes targeting different regions of a DNA sequence of interest.

In one embodiment, the method according to the invention comprises a capture-based approach such as developed by TWIST Bioscience (NGS Methylation Detection System) for DNA methylation analysis.

In a particular aspect, the method comprises step(s) of screening/capturing, aligning and/or clustering of DNA sequences and/or subsequences thereof. Such step(s) can be performed using DNA sequencing, by any methods known in the art such as for example massive parallel sequencing (e.g., next generation sequencing (NGS)), sequencing-by-synthesis, real-time (e.g., single-molecule) sequencing, bead emulsion sequencing, nanopore sequencing. Quantitative polymerase chain reaction (qPCR) (e.g., methylation sensitive restriction enzyme quantitative polymerase chain reaction or MSRE-qPCR) can also be used.

Preferably, the clustering of DNA subsequences is based on DNA nucleotide sequence similarity or identity and/or on similar sequence lengths.

In an aspect, the clustering of the DNA subsequences is performed with the help of an algorithm, in particular using vsearch. The subsequence is added to the cluster if the pairwise identity with the centroid is higher than 0. The pairwise identity is defined as the number of (matching columns)/(alignment length).

In an embodiment, the total number of clusters is chosen dynamically; so that the DNA subsequences are clustered in n clusters, n being defined dynamically depending on the size of said clusters, a cluster being defined as reference sequences representing at least 15% of the total sequences.

In a particular embodiment, the DNA subsequences are clustered in at least 1, 2, 3, 4, 5, 6, 7, 8, 9, 10, 11, 12, 13, 14, 15, 16, 17, 18, 19 or 20 clusters, for example in between 5 and 15 clusters or 5 and 10 clusters, preferably in 10 clusters. The number of clusters to take into account can particularly be based on the percentage of total reads a given cluster represents.

In a particular embodiment, DNA subsequences are clustered on the basis of sequence identity. The resulting cluster comprises subsequences having at least about 50%, 60%, 70%, 80%, 85%, 90%, 95%, 97%, 98% or 99% sequence identity.

The comparison of sequences and determination of percent identity between two sequences can be accomplished using any methods known in the art and/or involve a computational algorithm, such as BLAST (basic local alignment search tool).

The method according to the invention preferably comprises a step of selection of a reference sequence for (and in) each cluster. The reference sequence can be one or more sequence(s) selected from the cluster(s).

Preferably, the reference sequence is the most represented, or is a representative, sequence of the considered cluster. By most represented or representative sequence, it is meant a sequence which represents the maximum number of sequences that have at least 60%, at least 70% or at least 80% sequence identity similar to a consensus size/sequence found within the cluster.

In particular, the reference sequence is not a reference genome. The method thus comprises aligning sequencing data from repetitive sequences without using a reference genome.

Preferably, the reference sequence is the sequence having the longest nucleic acid length in the cluster. In an embodiment where the method is a PCR-based method, the longest nucleic acid length is the length of the sequence of the insert between the forward and reverse primer optionally with a margin error of +/−15 nucleic acids, preferably +/−10 nucleic acids, even more preferably +/−5 nucleic acids.

In a preferred embodiment wherein the total number of clusters is chosen dynamically so that the DNA subsequences are clustered in n clusters, n being defined dynamically depending on the size of said clusters, the reference sequences are the centroids of the n largest clusters (i.e., clusters comprising the largest number of sequences). The centroid of sequences is the center sequence which minimizes the sum of distances to all sequences in the cluster.

Preferably, the method comprises the selection of the largest clusters (i.e., the cluster comprising the higher number of DNA sequences compared to the total number of DNA sequences).

In particular, such cluster includes a number of DNA subsequences that represents a minimum of at least 10%, 15%, 20%, 25% or 30% of the total number of DNA subsequences.

In the context of the herein described method, at least 1, 2, 3, 4, 5, 6, 7, 8, 9, 10, 11, 12, 13, 14, 15, 16, 17, 18, 19 or 20 reference sequence(s) is/are identified for each cluster.

Preferably, between 5 and 20, between 5 and 15 or between 5 and 10 reference sequences, between 1 and 20, between 1 and 15 or between 1 and 10 reference sequences are defined per cluster. Even more preferably, 10 reference sequences are chosen/selected/identified/determined.

In an embodiment, 1 sequence of reference is defined per cluster and 10 sequences total from the 10 biggest clusters are used as reference sequences.

The method according to the invention preferably comprises a step of aligning (each of) the reference sequences of (each) the clusters by allowing the alignment on positions of CpG dinucleotides.

The reference sequences are thus aligned with each other to obtain a pool of reference sequences. The pool of reference sequences constitutes a reference allowing the alignment all of the sub-sequences.

Preferably, the alignment of the remaining sub-sequences on the selected reference sequences is based on a score that favors in order: the alignments of G/G, then T/T, C/C, C/T and T/C, and finally the other nucleotides of the sequence. The score can be established by using for example program mafft (preferably with the following parameters: --textmatrix <custom_score_matrix.txt> --retree 2). The reference sequences are preferably aligned pairwise using a custom score matrix to favor dinucleotides CG/TG alignment over other possible dinucleotides combinations (e.g., AG/AC/TC).

This step aims to confirm positions of CpG dinucleotides in reference sequences. In particular, alignment of the DNA sequences or subsequences thereof from healthy subjects aims at confirming the positions of CpG dinucleotides.

The method preferably comprises a step of aligning the remaining sub-sequences of the clusters on the selected reference sequences.

In a particular embodiment, the method comprises a step of aligning all subsequences, in particular all amplified subsequences to the reference sequence(s). This step makes it possible to further check the quality of the DNA subsequences alignment.

Particularly, the reads are aligned onto the reference sequence(s) identified, using an algorithm having a time complexity of O(n) (n being the number of reads to align), allowing the alignment of millions of reads.

An algorithm is said to have a linear time complexity when the running time increases linearly with the length of the input.

In a particular, optional embodiment, the method comprises an additional step of checking the presence of (sufficient) CpG dinucleotide sites susceptible to be methylated in each reference sequence, which is performed either before or after the alignment step of the clusters' reference sequences, preferably after.

In particular, at least 20% of CG+TG dinucleotides within a dinucleotide site are selected and this site preferably has at least 5% of CG dinucleotides (in particular after bisulfite sequencing thus representing methylated CG). This step can be referred herein as “CG calling”. Preferably, the method comprises a step of confirmation of CpG sites that are methylated or susceptible to be methylated.

Preferably, the reference sequence comprises at least 1, 2, 3, 4, 5, 6, 7, 8, 9, 10, 20, 30, 60 or 90 CpG dinucleotides, preferably at least 30 or 90 CpG dinucleotides.

Determination of the presence of CpG dinucleotide sites susceptible to be methylated in each reference sequence, is preferably performed between the step d) and e) of the above-mentioned method or during step e) of the above-mentioned method.

Alternatively, determination the presence of CpG dinucleotide sites susceptible to be methylated in each reference sequence, can be performed between the step b) and c) of the above mentioned method.

In a particular embodiment, the determination of CpG dinucleotide sites susceptible to be methylated is performed on DNA from a healthy subject or population of healthy subject, in particular to avoid biases related to cancer hypomethylation.

Once the reference sequences have been aligned, it is possible to easily identify CpG sites, and check (for confirmation) that a particular CpG site can be methylated, by comparing all of the subsequences that have been aligned. In particular, dinucleotides CG and TG are identified in each of the subsequences.

At a particular dinucleotides position, the respective percentage of dinucleotides CG and of dinucleotides TG of a particular dinucleotide position in the set of subsequences is calculated (number of CG and number of TG of all the subsequences).

For a particular dinucleotide position in the sequence, a percentage of CG superior to 5% combined with a percentage of CG+TG superior to 20% is indicative of a CpG site susceptible to be methylated, in particular after bisulfite sequencing, thus representing methylated CG.

A percentage of TG superior to 95% is indicative of a dinucleotide site that is not susceptible to be methylated, in particular recurrently methylated.

A percentage of CG+TG inferior to 20% is indicative of a dinucleotide site that is not susceptible to recurrently be a proper template for methylation.

The method according to the invention preferably comprises a step of determining the CpG methylation status of each sub-sequence by determining at each CpG site of the sub-sequence if the CpG dinucleotide is methylated or not, thereby determining a CpG methylation profile comprising a CpG methylation level and/or a proportion of CpG methylation haplotypes of the sub-sequences.

The methylation status and the methylation profile can be assessed by a variety of methods known in the art and/or by methods provided herein. Methods of measuring methylation status may involve, for example and without limitation, whole genome sequencing, targeted enzymatic methylation sequencing, methylation-status-specific polymerase chain reaction (PCR), mass spectrometry, methylation arrays, methylation-specific nucleases, mass-based separation, target-specific capture, and/or methylation-specific oligonucleotide primers. A particular method for assessing methylation utilize a bisulfite reagent (e.g., sodium bisulfite) or an enzymatic conversion reagent (e.g., Tet methylcytosine dioxygenase 2).

As used herein, “methylation status” or “methylation state” refer to the fact that a CpG dinucleotide is methylated or not.

As used herein, “methylation profile” refers to the number, frequency, or pattern of methylation at CpG methylation sites within a sequence of interest, in particular a sequence of interest within a DNA sequence. Accordingly, a change of the methylation profile between a first state and a second state can be, or include, an increase in the number, frequency, or pattern of methylated CpG sites, or can be, or include, a decrease in the number, frequency, or pattern of methylated sites. In various instances, a change in the methylation status is a change in the methylation level and/or methylation pattern.

Preferably, the CpG methylation profile comprises a CpG methylation level, in particular methylation levels at each CpG sites and/or proportions of CpG methylation haplotypes of the sub-sequences.

As used herein, the term “methylation level” or “methylation value” refers to a numerical representation of a methylation status, e.g., a number that represents the frequency or ratio of methylation of CpG sites in a subset of sequences of interest. In a particular embodiment, the methylation level is the percentage of CpG that is methylated in a subset of DNA sequences. It means that in a cluster of subsequences, at a particular CpG dinucleotide, the methylation status is determined for such position in each of the sub-sequences of the cluster. Then, a ratio may be established as follows: (number of methylated CpG dinucleotides at a particular dinucleotide position)/(total number of sub-sequences in the cluster). Preferably, the methylation level at a particular CpG site in a cluster of subsequences is the proportion of CG dinucleotides over the count of CG+TG dinucleotides, over the count of CG+TG+TA dinucleotides (CG/CG+TG/CG+TG+TA).

As interchangeably used herein, the terms “methylation pattern”, “methylation motifs”, “methylation signature” or “methylation haplotype” refers to a numerical representation of a “methylation profile”, e.g., a number that represents a unique succession of a string/chain of methylated cytosines, at single-molecule resolution, thereby creating a unique motif. This particularly refers to methylation state of successive CpG sites within each sequence (or subsequence or amplicon).

In a particular embodiment, when a cytosine is methylated, the number attributed is “1” whereas a cytosine that is not methylated has the number “0”. Thus, in a sequence of interest, the succession of cytosines methylated or unmethylated results in a succession of numbers “1” or “0”. Such alternance of 0 and 1 provides a particular methylation pattern to the studied DNA subsequence.

The “proportion of methylation haplotypes” refers to the proportion or percentage of a particular methylation pattern among a population of sequences, in particular in a cluster of subsequences.

For example, if the sequence of interest comprises two CpG sites, then, the methylation haplotype is: 0-0 (both CpG sites unmethylated), 1-1 (both sites methylated), 1-0 (first CpG site methylated) or 0-1 (second CpG site methylated). The proportion of methylation haplotype in a cluster of sub-sequences is the percentage of each of the haplotype 0-0, 1-1, 1-0, 0-1 among all the sub-sequences of the cluster.

In a particular embodiment, some, or each, of steps a) through f) of the herein described method are performed with another set of sub-sequences of interest. The other set may be completely distinct/different or at least partly distinct from the originally (or any previously) used set.

In a particular aspect, and for a particular DNA sequence, 1 subsequence or at least 2, 3, 4, 5, 6, 7, 8, 9, 10, 11, 12, 13, 14, 15, 16, 17, 18, 19, 20, 25, 30, or 35 subsequences, can be separately or simultaneously analyzed with respect to its/their methylation profile.

The subsequences may be overlapping subsequences in a DNA of interest. In particular, when aligned to the complete DNA sequence, the subsequences may overlap with each other by between 5 and 100 bp, in particular between 5 and 25 bp in 3′ or 5′.

Preferably, the subsequences can be defined such as covering the entire repeated sequence of interest.

Alternatively, the subsequences can be defined such as covering only part(s) of the repeated sequence of interest. Preferably, the subsequences comprise a DMR, particularly a region that is differentially methylated in cancer compared to a healthy condition.

In an embodiment, the repeated sequence is LINE-1 and the method comprises at least 4, 5, 6, 7, 8, 9,10, 20, 50, 100, 200, 250 or 300 sub-sequences of interest.

In an embodiment, the repeated sequence is LINE-1 and the method comprises between 5 and 10 sub-sequences of interest, more preferably between 7 and 9 sub-sequences of interest, even more preferably 8 sub-sequences of interest, and the method for determining the CpG methylation profile is performed for each of the sub-sequences, e.g., for eight subsequences.

In an embodiment, the repeated sequence is LINE-1 and the method comprises between 100 and 300 sub-sequences of interest, more preferably between 200 and 300 sub-sequences of interest, even more preferably 250 sub-sequences of interest, and the method for determining the CpG methylation profile is performed for each of the sub-sequences, e.g., for 250 subsequences.

Preferably, a subsequence of interest comprises, or consists essentially of, a sequence selected from SEQ ID NO: 3, 4, 5, 6, 7, 8, 9, 10, and any sequence having at least 85, 90, 95, 98, or 99% sequence identity thereto.

Each of the subsequence may be amplified with a pair of primer, in particular a pair of primers selected from the group comprising, in particular consisting of:
i) a forward primer having a sequence as set forth in SEQ ID NO: 11 and a reverse primer having a sequence as set forth in SEQ ID NO: 12; in particular to target amplicon #1 preferably such as described in SEQ ID NO: 3;
ii) a forward primer having a sequence as set forth in SEQ ID NO: 13 and a reverse primer having a sequence as set forth in SEQ ID NO: 14; in particular to target amplicon #2 preferably such as described in SEQ ID NO: 4;
iii) a forward primer having a sequence as set forth in SEQ ID NO: 15 and a reverse primer having a sequence as set forth in SEQ ID NO: 16; in particular to target amplicon #3 preferably such as described in SEQ ID NO: 5;
iv) a forward primer having a sequence as set forth in SEQ ID NO: 17 and a reverse primer having a sequence as set forth in SEQ ID NO: 18; in particular to target amplicon #4 preferably such as described in SEQ ID NO: 6;
v) a forward primer having a sequence as set forth in SEQ ID NO: 19 and a reverse primer having a sequence as set forth in SEQ ID NO: 20; in particular to target amplicon #5 preferably such as described in SEQ ID NO: 7;
vi) a forward primer having a sequence as set forth in SEQ ID NO: 21 and a reverse primer having a sequence as set forth in SEQ ID NO: 22; in particular to target amplicon #6 preferably such as described in SEQ ID NO: 8;
vii) a forward primer having a sequence as set forth in SEQ ID NO: 23 and a reverse primer having a sequence as set forth in SEQ ID NO: 24; in particular to target amplicon #7 preferably such as described in SEQ ID NO: 9;
viii) a forward primer having a sequence as set forth in SEQ ID NO: 25 and a reverse primer having a sequence as set forth in SEQ ID NO: 26; in particular to target amplicon #8 preferably such as described in SEQ ID NO: 10;
and any combination thereof.

A primer as described herein above may comprise adapter(s) such as Unique Molecular Identifiers (UMIs) or unique dual indexes (UDI). The primer may further comprise common or universal sequence(s) CS1 and/or CS2. Common sequence(s) can for example be common sequence 1 (CS1) (5′-ACACTGACGACATGGTTCTACA-3′ SEQ ID NO: 27) and/or common sequence 2 (CS2) (5′-TACGGTAGCAGAGACTTGGTCT-3′ SEQ ID NO: 28) universal primer sequences.

In a particular embodiment, the method comprises a step of identifying the amplicons before the clustering step. Such an identification may be performed in particular with the sequence of the primers and probes used

In a particular embodiment, the method comprises repeating one or several times some, or each, of steps a) through e), and optionally performing an additional step of comparing the CpG methylation profiles to each other in order to provide optimized CpG methylation profile(s).

The determination of the CpG methylation profile of a DNA sequence of interest gives insight on the status of (e.g., healthy or cancerous) the DNA sequence, and eventually of the subject from which the DNA sequence originates.

In a particular and preferred aspect, the invention makes it possible to distinguish a “healthy CpG methylation profile” from a “cancerous CpG methylation profile”.

As used herein, the term “healthy CpG methylation profile” refers to a CpG methylation profile that is correlated or indicative of a subject that is healthy, in particular who does not suffer from cancer.

As used herein, the term “cancerous CpG methylation profile” refers to a CpG methylation profile that is correlated with cancer or indicative that a subject suffers from cancer.

In some embodiments, the term “cancerous CpG methylation profile” refers to:
a CpG methylation profile that is correlated with a particular cancer origin/type or is indicative that a subject suffers from cancer of a particular origin/type (the origin of the cancer being for example, breast, colon or brain cancer);
a CpG methylation profile that is correlated with a particular stage of cancer or is indicative that a subject suffers from cancer of a particular stage (the stage of cancer being for example stage I, II or III); and/or
a CpG methylation profile that is correlated with cancer metastasis or is indicative that a subject suffers from cancer metastasis.

In some embodiments, the term “cancerous CpG methylation profile” refers to:
a CpG methylation profile that is correlated with a particular cancer origin/type and metastasis or is indicative that a subject suffers from cancer of a particular origin/type and metastasis (the origin of the cancer being for example, breast, colon or brain cancer);
a CpG methylation profile that is correlated with a particular stage of cancer and metastasis or is indicative that a subject suffers from cancer of a particular stage (the stage of cancer being for example stage I, II or III) and metastasis; or
a CpG methylation profile that is correlated with a particular cancer origin/type and stage or is indicative that a subject suffers from cancer of a particular origin/type and stage.

In a particular and preferred aspect, a herein described method is a partially or fully computer-implemented method.

### Training Methods

In another aspect, the invention concerns a method, in particular a computer-implemented method, of training a classifier for accurately distinguishing between a healthy CpG methylation profile and a cancerous CpG methylation profile, or for determining the health status of a subject, in particular for accurately distinguishing between a healthy subject and a subject suffering from cancer or between different types of cancer. These training methods rely in particular on the determination of CpG methylation profiles of DNA sequences of interest or sub-sequence(s) thereof, preferably with a method for determining a CpG methylation profile as herein described, such as under the paragraph “Determination of CpG methylation profile” appearing herein above. Thus, the herein above described aspects and embodiments in relation with the determination of the CpG profile may apply to any of the training methods described herein below.

Examples of cancers to which the methods according to the invention can refer are herein described in particular under the paragraph “Subject and biological sample”.

As used herein, the term “classifier” refers to a computer-implemented algorithm that performs classification, i.e. that can determine a likelihood score or a probability that an object classifies within a group of objects (e.g., a group of healthy CpG methylation profiles) as opposed to one or several other groups of objects (e.g., a group of cancerous CpG methylation profiles), and that maps said input object to a category (e.g. healthy or cancerous CpG methylation profiles). The term “classifier” may refer to one or multiple classifiers. For example, multiple classifiers may be trained, which may process data in parallel and/or as a pipeline. For example, output of one type of classifier (e.g., from intermediate layers of a neural network) may be fed as input into another type of classifier.

Examples of classifiers that can be used in the context of the present invention include for example, but are not limited to, artificial neural networks of various architectures (e.g., deep, convolutional, fully connected) and supervised machine learning classifiers such as Support Vector Machine (SVM) classifier, random forest classifier, decision tree classifier, K-nearest neighbor classifier (KNN), logistic regression classifier, nearest neighbor classifier, Gaussian mixture model (GMM), and nearest centroid classifier. It is not an exhaustive list and the skilled person in the art will be able to identify similar algorithms that can be equally used, although they are not specifically mentioned here. Details and rules of functioning of the mentioned algorithms have already been widely described in the literature. An important contribution is the set of input data provided to the classifier (i.e. a set of CpG methylation profiles or preprocessed information obtained from a set of CpG methylation profiles). Based on this input data, it is possible to create a suitable model using any appropriate supervised machine learning techniques. The selection of appropriate algorithms is therefore of secondary nature and can be carried out in many different ways and in various combinations obvious to those skilled in the art.

Preferably, the classifier is selected from Support Vector Machine (SVM) classifier, random forest (RF) classifier, decision tree classifier, K-nearest neighbor classifier (KNN), logistic regression classifier, nearest neighbor classifier, Gaussian mixture model (GMM), nearest centroid classifier and artificial neural networks such as deep, convolutional, fully connected neural networks. More preferably, the classifier is selected from Support Vector Machine (SVM) classifier, random forest (RF) classifier and neural networks, in particular convolutional neural network (CNN). Even more preferably, the classifier is random forest classifier.

A classifier utilizes some training data to understand how given input objects belong to a category/class or another. The classifier may be provided with a training set of biological samples from subject, such as a healthy and/or cancerous subject, said training set comprising DNA sequences, in particular cfDNA sequences, exhibiting features of healthy or cancerous CpG methylation profiles. Alternatively, the classifier may be provided with preprocessed information obtained from such a training set of DNA sequences.

In an aspect, the invention concerns a method, typically a computer implemented method, of training a classifier for accurately distinguishing between a healthy CpG methylation profile and a cancerous CpG methylation profile, said method comprising:
a) providing a training set of CpG methylation profiles of DNA sequences of interest or sub-sequences thereof, said DNA sequences of interest being repeated and distributed throughout a genome and comprising high density of CpG dinucleotides, or preprocessed information obtained from said training set of CpG methylation profiles of DNA sequences of interest or sub-sequences thereof, as an input to a classifier, said training set of CpG methylation profiles comprising CpG methylation profiles of DNA sequences, or of sub-sequences thereof, from subjects identified as healthy subjects and from subjects identified as cancerous subjects; and,
b) generating an output of the classifier for each CpG methylation profile input of DNA sequence of interest or sub-sequences thereof, said output classifying the CpG methylation profile input of DNA sequence of interest or sub-sequences thereof as a healthy CpG methylation profile or as a cancerous CpG methylation profile;
c) evaluating the classifier's performance for distinguishing between a healthy CpG methylation profile and a cancerous CpG methylation profile,
wherein the CpG methylation profile comprises a CpG methylation level and/or proportions of CpG methylation haplotype of the DNA sequence or sub-sequences thereof.

The invention also concerns a method, typically a computer-implemented method, of training a classifier for determining the health status of a subject, in particular for accurately distinguishing between a healthy subject and a subject suffering from cancer, said method comprising:
a) providing a training set of CpG methylation profiles of DNA sequences of interest or sub-sequences thereof, said DNA sequences of interest being repeated and distributed throughout a genome and comprising high density of CpG dinucleotides, or preprocessed information obtained from said training set of CpG methylation profiles of DNA sequences of interest or sub-sequences thereof, as an input to a classifier, said training set of CpG methylation profiles comprising CpG methylation profiles of DNA sequences, or sub-sequences thereof, from subjects identified as healthy subjects and from subjects identified as cancerous subjects; and,
b) generating an output of the classifier for each CpG methylation profile input of DNA sequence of interest or sub-sequences thereof, said output classifying the CpG methylation profile input of DNA sequence of interest or sub-sequences thereof as a healthy CpG methylation profile or as a cancerous CpG methylation profile;
c) classifying a subject with a healthy CpG methylation profile as a healthy subject and a subject with a cancerous CpG methylation profile as a subject suffering from cancer;
d) evaluating the classifier's performance for distinguishing between a healthy subject and a subject suffering from cancer,
wherein the CpG methylation profile comprises a CpG methylation level and/or a CpG methylation haplotype of the DNA sequence or sub-sequences thereof;
Step c) or d) of the herein above described training methods relates to the evaluation of the classifier's performance for distinguishing between i) healthy CpG methylation profile or subject and ii) cancerous CpG methylation profile or subject.

In a particular and preferred aspect, the CpG methylation profile of the DNA sequences of interest, or that of a sub-sequence thereof, is determined with a method for determining a CpG methylation profile as herein described, in particular under the paragraph “Determination of CpG methylation profile”.

In an embodiment, the method for determining a CpG methylation profile, comprises an alignment of the DNA sequences of interest of subset thereof from healthy subjects for confirming the positions of CpG dinucleotides susceptible to be methylated. Preferably, heavily methylated CpG sites are determined.

Alternatively or additionally, such method may further comprise an alignment of the DNA sequences of interest of subset thereof from cancerous subjects for confirming the positions of CpG dinucleotides susceptible to be methylated. Preferably, unmethylated CpG sites are determined.

In a particular embodiment, the evaluation of the classifier's performance carried out in the context of the invention is based on the classification of CpG methylation profiles of the DNA sequence of interest or sub-sequences thereof using a test set comprising CpG methylation profiles of DNA sequences or sub-sequences thereof from healthy subjects and cancerous subjects, said test set being distinct from the training set, the healthy or cancerous status of each subject of the test set and the training set being known, and CpG methylation profiles of DNA sequences or sub-sequences thereof of the test set being obtained and processed using the same method as that used to obtain and process CpG methylation profiles of DNA sequences or sub-sequences thereof with the training set.

Biological and clinical data from healthy and cancerous patients can easily be retrieved from clinical trials. Collaborations, such as the NIH Collaboratory Distributed Research Network provides mediated or collaborative access to clinical data repositories by eligible researchers. Additionally, the UW De-identified Clinical Data Repository (DCDR) and the Stanford Center for Clinical Informatics allow for initial cohort identification. National Program of Cancer Registries (CDC) provides support for states and territories to maintain registries that provide high-quality data. Data collected by local cancer registries enable public health professionals to understand and address the cancer burden more effectively. Clinical data are also published under the European Medicines Agency (EMA).

The performance of the classifier may be assessed using any method known by the skilled person. For example, the classifier's performance may be assessed by precision, recall or F1 score.

The classifier is considered as a well-performing classifier to distinguish between healthy and cancerous conditions if false positives are minimal.

To increase the classifier's performance, the training method, i.e. steps a) to c) or steps a) to d) (depending on the method described herein above which is the one considered), may be reiterated with some modifications such as increasing the number of healthy and cancerous CpG methylation profile in the training set of DNA sequences, using a distinct training set of DNA sequences. Another possibility of increasing the classifier's performance is to increase the number of sets of subsequences of cfDNA of interest used.

In a particular embodiment, the performance of the classifier is the classifier's accuracy. Accuracy of the classifier is the measure of correct prediction of the classifier compared to its overall data points. It is particularly the ratio of the units of correct predictions on the total number of predictions made by the classifiers. It is preferably the rate of correct classifications, either for an independent test set, or using cross-validation.

In a particular embodiment, the performance of the classifier is expressed/computed as an AUROC (Area Under the curve of the Receiver Operating Characteristic), an AUC (Area Under the Curve) or ROC (Receiver Operating Characteristic) Curve. ROC curve can be used to select a threshold for a classifier, which maximizes the true positives and in turn minimizes the false positives. The higher the AUC, it is assumed that the better the performance of the model at distinguishing between the positive and negative classes.

For example, for evaluating the classifier's performance, the true and false positive rates are evaluated at each run, with interpolation to generate all points of a ROC curve. At the end of the runs, an average ROC curve is generated and the 95% confidence interval is calculated based on the results of all runs. In the context of multiclass, the ROC curves of each class may be generated by taking the class under consideration as the positive class.

Preferably, the CpG methylation profiles of a DNA sequences of interest, or of sub-sequences thereof, is determined with a method for determining a CpG methylation profile as disclosed hereabove.

### Tests Methods

In another particular aspect, the invention concerns methods, in particular computer-implemented methods, for determining the health status of a subject, for determining if the subject is a healthy subject or a subject suffering from cancer, for determining the origin, or the stage, of a tumor, or monitoring the effect of/the response to an anti-cancer treatment/agent, for assessing the potency of a compound to revert a cancerous CpG methylation profile of a cfDNA sequence of interest from a subject suffering from cancer into a healthy CpG methylation profile, or for predicting the ability of a compound to treat a cancer. These tests methods particularly rely on the determination of the CpG methylation profile(s) of cfDNA sequence(s) of interest or sub-sequences thereof with a method for determining a CpG methylation profile as disclosed herein, in particular above under the paragraph “Determination of CpG methylation profile”. Thus, each and every aspect and embodiment described in relation with the determination of the CpG profile may apply to any of the test methods described herein below.

Examples of cancers envisioned by the tests methods according to the invention are particularly described here below under the paragraph “Subject and biological sample” and apply to any of the test methods described below.

Preferably, in the herein described test methods, the classifier has been trained using the herein described training method, in particular the training method describes herein above under the paragraph “Training methods”. Thus, each and every aspects and embodiment described in relation with the training method may apply to any of the test methods described herein below.

In a particular embodiment, the invention concerns an in vitro or in silico method of determining the health status of a subject, in particular of determining if the subject is a healthy subject or a subject suffering from cancer or cancer relapse, wherein the method comprises:
(i) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
(ii) using the classifier to identify the CpG methylation profile of the DNA sequence of interest or sub-sequences thereof of said subject as a healthy CpG methylation profile or as a cancerous CpG methylation profile as an output of the classifier.

In particular, the determination of the health status of the subject comprises the identification of CpG methylation profile of the DNA sequence of interest, or sub-sequences thereof, from said subject as a healthy CpG methylation profile or as a cancerous CpG methylation profile wherein a number of CpG methylation profiles classified as cancerous CpG methylation profile which is above a number of CpG methylation profiles classified as healthy CpG methylation profile, is indicative that said subject suffers from cancer, and/or a number of CpG methylation profiles classified as healthy CpG methylation profile which is above a number of CpG methylation profiles classified as cancerous CpG methylation profile, is indicative that said subject does not suffer from cancer.

In another embodiment, a number of CpG methylation profiles classified as healthy CpG methylation profile which is below a statistically significant threshold is indicative that said subject suffers from cancer, and/or a number of CpG methylation profiles classified as healthy CpG methylation profile which is equal to or above a statistically significant threshold is indicative that said subject does not suffer from cancer.

Alternatively, a number of CpG methylation profiles classified as cancerous CpG methylation profile which is above a prediction level threshold is indicative that said subject suffers from cancer, and/or a number of CpG methylation profiles classified as cancerous CpG methylation profile which is below a prediction level threshold is indicative that said subject does not suffer from cancer.

In an embodiment, the prediction level threshold is of at least 50%, 51%, 52%, 53%, 54%, 55%, 56%, 57%, 58%, 59% or 60%. Preferably, the prediction level threshold is of at least 50%. In particular, when the algorithm is random forest, the prediction level threshold is of at least 50%, e.g., when at least 50% of the decision trees consider the methylation profile as “cancerous” then the methylation profile is considered (classified) as “cancerous”.

Thus, in a particular embodiment, if the CpG methylation profiles (e.g. methylation level and/or methylation haplotype) of a DNA sequence of interest or sub-sequences thereof for a particular subject are considered as “healthy” in at least 51% of the cases (i.e. the number of runs performed with the statistical model, for example at least 1000, 5 000 or 10 000 runs), said subject is identified as a subject who does not suffer from cancer.

In another embodiment, if the CpG methylation profiles of a DNA sequence of interest or sub-sequences thereof from a subject are considered as “cancerous” in at least 50% of the cases (i.e. the number of runs performed with the statistical model), said subject is identified as a subject suffering from cancer.

In a particular embodiment, the prediction level threshold is determined or set dynamically. Varying the prediction score threshold used for classification allows an emphasis on sensitivity. It also allows for a finer tuning of each individual model by selecting a different threshold than the default 0.5 normally used for classification, in order to accurately classify more samples of a given class A without necessarily increasing the rate of misclassification of samples of a given class B.

Prediction level threshold can be easily set by the man skilled in the art depending on the data set, the type of cancer, or the type of response (e.g. healthy vs cancerous subject, origin of tumor etc.) In a particular embodiment, the method of determining the health status of a subject comprises the determination of the presence of a primary tumor, preferably an early-stage tumor (in particular a primary tumor of stage I, II or Ill), the presence of cancer relapse, or the presence of metastasis in the subject, if the classifier identifies the CpG methylation profile as a cancerous CpG methylation profile.

The method according to the invention is particularly useful in the early diagnosis of a cancer or the diagnosis of a cancer at an early stage (i.e., stage I or II or Ill).

Thus, in a particular aspect, the invention concerns an in vitro or in silico method of determining the stage of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
(b) using the classifier to:
i) identify CpG methylation profile of the DNA sequence of interest or sub-sequences thereof from said subject as a healthy CpG methylation profile or as a cancerous CpG methylation profile,
ii) compare the cancerous CpG methylation profile of the DNA sequence of interest or sub-sequences thereof from the subject to CpG methylation profiles of tumors of different known stages, in particular to CpG methylation profiles of tumors from populations of cancerous subjects having tumors of different known stages; and
iii) correlate the cancerous CpG methylation profile to a particular stage to determine the stage of the tumor from the subject as an output of the classifier.

The invention also concerns an in vitro or in silico method of determining the origin of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile from different tumors origins, and
(b) using the classifier to identify the CpG methylation profile of the DNA sequence of interest or sub-sequences thereof of said subject as a healthy CpG methylation profile or as a cancerous CpG methylation profile from a particular tumor origin to determine the origin of the tumor from the subject as an output of the classifier.

In an aspect, the origin of the tumor is the origin of a metastasis.

In another aspect, the method according to the invention can advantageously be used in the early detection of cancer relapse.

Additionally, or alternatively to the determination of the stage of the tumor, the method according to the invention may be used to determine the origin of a tumor, i.e. the type of the primary tumor, for example a colon cancer, breast cancer, lung cancer, etc.

Thus, the invention also concerns an in vitro or in silico method of determining the origin of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
(b) using the classifier to:
i) identify CpG methylation profile of the DNA sequence of interest or sub-sequences thereof from said subject as a healthy CpG methylation profile or as a cancerous CpG methylation profile,
ii) compare the cancerous CpG methylation profile of the DNA sequence of interest or sub-sequences thereof from the subject to CpG methylation profiles of tumors of different known origins, in particular to CpG methylation profiles of tumors from populations of cancerous subjects having tumors of different known origins; and
iii) correlate the cancerous CpG methylation profile to a particular origin to determine the origin of the tumor from the subject as an output of the classifier.

The invention also concerns an in vitro or in silico method of determining the stage of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile of different stages, and
(b) using the classifier to identify the CpG methylation profile of the DNA sequence of interest or sub-sequences thereof of said subject as a healthy CpG methylation profile or as a cancerous CpG methylation profile of a particular stage to determine the stage of the tumor from the subject as an output of the classifier.

The invention also concerns an in vitro or in silico method of determining the origin and the stage of a tumor from a subject, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile from different tumors origins and stages, and
(b) using the classifier to identify the CpG methylation profile of the DNA sequence of interest or sub-sequences thereof of said subject as a healthy CpG methylation profile or as a cancerous CpG methylation profile from a particular tumor origin and stage to determine the origin and stage of the tumor from the subject as an output of the classifier.

The methods according to the invention is useful in the diagnosis of primary tumor and metastasis. Even if metastasis generally spread from a primary tumor, over 10% of patients presenting to oncology units have metastases without a primary tumor found.

Thus, the invention also concerns an in vitro or in silico method of determining if a subject suffers from cancer metastasis, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
(b) using the classifier to:
i) identify CpG methylation profile of the DNA sequence of interest or sub-sequences thereof from said subject as a healthy CpG methylation profile or as a cancerous CpG methylation profile,
ii) compare the cancerous CpG methylation profile of the DNA sequence of interest or sub-sequences thereof from the subject to known CpG methylation profiles of metastasis, in particular to CpG methylation profiles of tumors determined/obtained from populations of cancerous subjects known to have metastasis (for example with a method as herein described); and
iii) correlate the cancerous CpG methylation profile to a known cancer metastasis CpG methylation profile to determine if the subject suffers from cancer metastasis as an output of the classifier.

The invention also concerns an in vitro or in silico method of determining if a subject suffers from cancer metastasis, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, said cancerous CpG methylation profile being related to metastasis and
(b) using the classifier to identify the CpG methylation profile of the DNA sequence of interest or sub-sequences thereof of said subject as a healthy CpG methylation profile or as a cancerous metastatic CpG methylation profile to determine if the subject suffers from cancer metastasis as an output of the classifier.

The invention also concerns an in vitro or in silico method of determining the stage of a tumor from a subject, wherein the determination of the stage of the tumor comprises the determination of the presence of metastasis, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile of different stages including metastasis, and
(b) using the classifier to identify the CpG methylation profile of the DNA sequence of interest or sub-sequences thereof of said subject as a healthy CpG methylation profile or as a cancerous CpG methylation profile of a particular stage including metastasis to determine the stage of the tumor and the presence of metastasis as an output of the classifier.

The invention also concerns an in vitro or in silico method for determining the origin of a tumor from a subject and if the subject suffers from metastasis, wherein the method comprises:
(a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and metastatic cancerous CpG methylation profile from different tumors origins, and
(b) using the classifier to identify the CpG methylation profile of the DNA sequence of interest or sub-sequences thereof of said subject as a healthy CpG methylation profile or as a metastatic cancerous CpG methylation profile from a particular tumor origin to determine the origin of the tumor from the subject and to determine if the patient suffers from metastasis as an output of the classifier.

The methods of determining if a subject suffers from cancer according to the invention (i.e., if the patient is cancerous or healthy) can be followed by methods for determining the origin of the cancer, for determining the stage of the cancer and/or for determining if the patient suffers from metastasis, in particular if the subject has been classified as a cancerous subject.

The tests method of determining if a subject suffers from cancer according to the invention may be performed once or several time during a subject's lifetime. Thus, it is possible to monitor the occurrence of a cancer, the evolution of a cancer, or the occurrence of a cancer or metastatic relapse.

In a particular embodiment, the subject suffering from cancer is a subject having received/been exposed to an anti-cancer treatment such as resection surgery, chemotherapy, radiotherapy or immunotherapy.

In a particular embodiment, the DNA from the subject is provided once to determine if the subject suffers from cancer, then once or several times during the first line of treatment if the subject suffers from cancer, in particular to determine if the treatment is effective, e.g. if the subject is cured or not from cancer or if symptoms related to cancer are alleged or not.

The methods of determining if a subject suffers from cancer according to the invention may also be performed after a first line of treatment or after the patient has been considered as cured from cancer, e.g., six months, one year, two years, three years, four years, five years, or ten years after the first line of treatment, or after the patient has been identified/considered as cured.

This advantageously allows to monitor the occurrence of cancer or metastatic relapse. If such event occurs, a second line of treatment may be administered to the subject. The DNA from the subject may be provided once or several times during the second line of treatment.

The efficiency of the first and/or second lines of treatment may be assessed by a method of monitoring the response to an anti-cancer treatment, in particular to a therapeutic compound, according to the invention. Such method may be performed once or several times during the first and/or second line of treatment.

Thus, the invention may concern an in vitro or in silico method of monitoring the response to an anti-cancer treatment, in particular to a therapeutic compound/agent, of a subject suffering from cancer, wherein the method comprises:
(i) providing at least one DNA sequence of interest or sub-sequences thereof from a first liquid biopsy from a subject suffering from cancer before the administration of the therapeutic compound to the subject as a first input, said DNA sequence of interest being repeated through the subject genome and comprising high density of CpG sites or a fragment thereof, or preprocessed information obtained from said first liquid biopsy, and a second liquid biopsy comprising at least one DNA sequence of interest or sub-sequences thereof from said subject after the administration of a therapeutic compound as a second input, or preprocessed information obtained from said second liquid biopsy, to a classifier trained to distinguish between DNA sequence having a healthy CpG methylation profile and DNA sequence having a cancerous CpG methylation profile; and
(ii) using the classifier to identify each CpG methylation profile of each DNA sequence of the first liquid biopsy as having a healthy CpG methylation profile or a cancerous CpG methylation profile as a first output of the classifier, and to identify each CpG methylation profile of each DNA sequence of the second liquid biopsy as having a healthy CpG methylation profile or a cancerous CpG methylation profile as a second output of the classifier, and
wherein a number of DNA sequence of interest classified as having a healthy CpG methylation profile in the second output of the classifier which is above a number of DNA sequence of interest classified as having a healthy CpG methylation profile in the first output of the classifier is indicative that the subject is responsive to said therapeutic compound, and wherein a number of DNA sequence of interest classified as having a healthy CpG methylation profile in the second output of the classifier which is equal to or below a number of DNA sequence of interest classified as having a healthy CpG methylation profile in the first output of the classifier is indicative that the subject does not respond to said therapeutic compound.

Alternatively, a number of DNA sequence of interest classified as having a cancerous CpG methylation profile in the second output of the classifier which is below a number of DNA sequence of interest classified as having a cancerous CpG methylation profile in the first output of the classifier is indicative that the subject is responsive to said therapeutic compound, whereas a number of DNA sequence of interest classified as having a cancerous CpG methylation profile in the second output of the classifier which is equal to or above a number of DNA sequence of interest classified as having a cancerous CpG methylation profile in the first output of the classifier is indicative that the subject does not respond (/is resistant) to said therapeutic compound.
it is possible to monitor the response to an anti-cancer treatment, in particular to a therapeutic compound/agent, of a subject suffering from cancer without the help of a machine learning approach, but by determining the CpG methylation profile of the patient before and after treatment, in particular following the methods of determination of CpG methylation profile as disclosed herein.

Preferably, the anti-cancer treatment is selected from the group consisting of resection surgery, chemotherapy, radiotherapy or immunotherapy.

Preferably, the therapeutic compound is a chemotherapeutic or immunotherapeutic compound. Chemotherapeutic compounds may be, without limitation, alkylating agents, antimetabolites, plant alkaloids, topoisomerase inhibitors, and antitumor antibiotics. Immunotherapeutic compounds may be for example and without limitation, antibodies, cytokines or interferons.

The method of monitoring the response to an anti-cancer treatment, in particular to a therapeutic compound of a subject suffering from cancer, relies on the change of cancerous CpG methylation profile of a DNA sequence of interest from a subject suffering from cancer into a healthy CpG methylation profile. This change into a healthy CpG methylation profile indicates the efficiency of the anti-cancer treatment in the considered subject.

In another embodiment, the method of monitoring the response to an anti-cancer treatment, in particular to a therapeutic compound of a subject suffering from cancer, relies on the change of CpG methylation level. For example, if a DNA sequence comprises CpG sites known to be hypomethylated in cancer, then a reduction of the hypomethylation indicates the efficiency of the anti-cancer treatment in the considered subject.

Thus, the invention also concerns an in vitro or in silico method of assessing the potency of a compound to revert a cancerous CpG methylation profile of a DNA sequence of interest from a subject suffering from cancer into a healthy CpG methylation profile, wherein the method comprises:
(i) providing a DNA sequence of interest or sub-sequences thereof from the subject having been treated with a compound, said DNA sequence of interest being repeated and distributed throughout the subject's genome and comprising high density of CpG dinucleotides or any fragment thereof, or preprocessed information obtained from said at least one DNA sequence of interest or sub-sequences thereof, as an input to a classifier trained to distinguish between DNA sequence having a healthy CpG methylation profile or a cancerous CpG methylation profile; and
(ii) using the classifier to detect DNA sequences having a healthy CpG methylation profile and/or DNA sequences having a cancerous CpG methylation profile as an output of the classifier, wherein an amount of DNA sequences having a healthy methylation profile above a reference amount of DNA sequences having a healthy methylation profile obtained from the subject before any treatment with the compound is indicative that the compound is able to revert the cancerous CpG methylation profile into a healthy CpG methylation profile.

The ability of a compound to revert a cancerous CpG methylation profile of a DNA sequence of interest from a subject suffering from cancer into a healthy CpG methylation profile may be indicative of the capacity of the compound to treat cancer.

Thus, the invention also concerns an in vitro or in silico method of predicting or testing the ability of a compound to treat a cancer. This method comprises a step of assessing the potency of a compound to revert a cancerous CpG methylation profile of DNA sequences of a subject into a healthy CpG methylation profile, typically with a method as disclosed herein, wherein an amount of DNA sequences classified as having a healthy CpG methylation profile in a biological sample of the subject who has been treated with the compound which is above the reference amount obtained from a biological sample of the subject before any treatment of said subject with said compound, is indicative that said compound is useful in the treatment of said cancer.

In a particular embodiment, the test methods disclosed herein rely on preprocessed information obtained from DNA sequence(s) of interest or sub-sequences thereof.

Preferably, in the test methods disclosed herein, samples are randomly drawn without replacement from the training data set. This means that the samples used for training the classifier are distinct (i.e., not the same) from the samples used in the Test methods according to the invention. For example, the population of samples is split into 60% for training the classifier, 40% for the testing methods.

Preferably, in the methods disclosed herein using a classifier, the classifier is selected from a Support Vector Machine (SVM) classifier, random forest (RF) classifier, decision tree classifier, K-nearest neighbor classifier (KNN), logistic regression classifier, nearest neighbor classifier, Gaussian mixture model (GMM), nearest centroid classifier, and an artificial neural network such as deep, convolutional, or fully connected neural network, more preferably selected from a Support Vector Machine (SVM) classifier, random forest (RF) classifier and convolutional neural network (CNN), and even more preferably is a RF classifier.

In a particular aspect, the method of the invention further comprises the determination of the presence of a mutation or genetic alteration in at least one gene deregulated in cancer such as one of the most commonly used alterations to detect ctDNA among the 299 recurrent oncogenic mutations identified from The Cancer Genome Atlas (TCGA) described in Bailey et al., Cell. 2018; 173(2):371-385.e18), particular, EGFR, TPp53, AKT1, BRAF, ERBB2, KIT, MET, RB1, FGFR, JAK, TSC, PDGFR, PIK3CA, ESR1, NRAS, CTNNB1, FBXW7, APC, CDKN2A, PTEN, FGFR2, HRAS, KRAS, PPP2R1A, GNAS (see for example Cohen, Science 1, eaar3247-10 (2018)) or any combination thereof.

Mutations in such genes have been well described in the art as to be associated with cancer.

In a further particular aspect, the herein described methods, in particular the training methods and tests methods, are computer-implemented methods.

In another particular aspect, the invention concerns a computing system comprising:
a memory storing at least one instruction of a classifier trained according to any of the training methods herein described, in particular a method of training a classifier for accurately distinguishing between a healthy CpG methylation profile and a cancerous CpG methylation profile or between a healthy subject and a subject suffering from cancer; and
a processor accessing to the memory for reading the aforesaid instruction(s) and executing the test methods of the invention, in particular a method for determining the health status of a subject, in particular a method for determining if a subject is a healthy subject or a subject suffering from cancer or cancer relapse, for determining the origin of a tumor from a subject, for determining the stage of a tumor, for monitoring the response to a therapeutic compound of a subject suffering from cancer, or for assessing the potency of a compound to revert a cancerous CpG methylation profile of a DNA sequence of interest from a subject suffering from cancer into a healthy CpG methylation profile, or for assessing the potency of a compound to treat cancer.

### Kit

In another aspect, the invention concerns a kit of primers or probes targeting a DNA sequence, preferably DNA sequence from a subject encoding a repeated sequence distributed throughout the subject's genome, and preferably comprising high density of CpG dinucleotides, even more preferably a retrotransposon.

In particular, the primers or probes target a DNA encoding a LINE-1 retrotransposon, preferably a LINE-1 retrotransposon such as described under SEQ ID NO: 2 or 29 or having at least 85%, 90%, 95%, 97%, 98%, or 99% sequence identity thereto.

Such primer or probe can be complementary of any region of the LINE-1 retrotransposon, such as for example regions of the 5′UTR, ORFI, ORFII or 3′UTR.

In a particular embodiment, the primers targeting LINE-1 comprise adapter(s) such as Unique Molecular Identifier(s) (UMI(s)) or unique dual index(es) (UDI(s)).

In a particular embodiment, the primers comprise common or universal sequence(s) CS1 and/or CS2. A common sequence can for example consists of, or comprise, common sequence 1 (CS1) (5′-ACACTGACGACATGGTTCTACA-3′ SEQ ID NO: 27) and/or common sequence 2 (CS2) (5′-TACGGTAGCAGAGACTTGGTCT-3′ SEQ ID NO: 28) universal primer sequences.

In a very particular embodiment, the kit according to the invention may comprise probes of between 100 and 150 bp, preferably 120 bp, which start every 20 to 30 bp, preferably every 24 bp. Preferably, such probes are design such as to cover at least 75%, 80%, 85%, 90% or 95% of the DNA sequence of interest.

In another embodiment, the kit according to the invention may comprise primers of between 20 and 100 bp. Such probes are design such as to cover at least 5% of the DNA sequence of interest.

A particular kit according to the invention comprises at least 4 primers or probes selected from the group of primers or probes having respectively a sequence as set forth in SEQ ID NO: 11, 12, 13, 14, 15, 16, 17, 18, 19, 20, 21, 22, 23, 24, 25 or 26 or a sequence having at least 80%, 85%, 90%, 95%, 97%, 98% or 99% identity thereto.

In a particular embodiment, the invention concerns a kit of primer pairs targeting sub-sequences of a DNA sequence encoding a LINE-1 retrotransposon, preferably a LINE-1 retrotransposon such as described under SEQ ID NO: 1, said kit comprising at least 4 primer pairs selected from the group consisting of:
i) a forward primer having a sequence as set forth in SEQ ID NO: 11 and a reverse primer having a sequence as set forth in SEQ ID NO: 12;
ii) a forward primer having a sequence as set forth in SEQ ID NO: 13 and a reverse primer having a sequence as set forth in SEQ ID NO: 14;
iii) a forward primer having a sequence as set forth in SEQ ID NO: 15 and a reverse primer having a sequence as set forth in SEQ ID NO: 16;
iv) a forward primer having a sequence as set forth in SEQ ID NO: 17 and a reverse primer having a sequence as set forth in SEQ ID NO: 18;
v) a forward primer having a sequence as set forth in SEQ ID NO: 19 and a reverse primer having a sequence as set forth in SEQ ID NO: 20;
vi) a forward primer having a sequence as set forth in SEQ ID NO: 21 and a reverse primer having a sequence as set forth in SEQ ID NO: 22;
vii) a forward primer having a sequence as set forth in SEQ ID NO: 23 and a reverse primer having a sequence as set forth in SEQ ID NO: 24;
viii) a forward primer having a sequence as set forth in SEQ ID NO: 25 and a reverse primer having a sequence as set forth in SEQ ID NO: 26;
and any combination thereof.

Preferably, the kit comprises at least 5, 6, 7 or 8 primer pairs, even more preferably 8 primer pairs such as disclosed herein above.

The invention also concerns the use of a kit according to the invention, for amplifying sub-sequences of a DNA sequence encoding a LINE-1 retrotransposon, preferably a LINE-1 retrotransposon such as described under SEQ ID NO: 2 or 29, for the diagnosis of cancer, in particular by PCR multiplex.

### Subject and Biological Sample

A sample analyzed using methods and kits provided herein can be any biological sample and/or any sample including DNA. The sample is typically obtained from a subject.

In a particular embodiment, the DNA sequence of interest is a cfDNA. Cellular DNA methylation patterns are conserved in cell-free DNA (cfDNA).

cfDNA may be provided in a biological sample, for example a fluid sample obtained, from a subject. Indeed, cfDNA may be found in a biological fluid such as, e.g., plasma, serum, or urine. The concentration of cfDNA is typically low in a biological sample, but can significantly increase under particular conditions, including without limitation pregnancy, autoimmune disorder, myocardial infraction, and cancer. Circulating tumor DNA (ctDNA) is the component of circulating DNA specifically derived from cancer cells. cfDNA and ctDNA provide a real-time or nearly real-time metric of the methylation status of a source tissue. cfDNA and ctDNA have a half-life in blood of about 2 hours, such that a sample taken at a given time provides a relatively timely reflection of the status of a source tissue.

In another particular embodiment, the DNA sequence of interest is a circulating tumor DNA (ctDNA). ctDNA is a tumor-derived fragmented DNA present in the bloodstream that is not associated with cells.

As used herein, the term “subject” refers to an organism, typically a mammal (e.g., a human). In particular, the subject is suffering from a disease, disorder or (abnormal) condition. In a particular aspect, the subject is susceptible to/prone to develop a disease, disorder, or (abnormal) condition. In another particular aspect, the subject displays one or more symptoms or characteristics of a disease, disorder or condition. In another particular aspect, the subject is not suffering from a disease, disorder or (abnormal) condition, or does not display any symptom or characteristic of a disease, disorder, or condition i.e., the subject is a healthy subject. In another aspect, the subject is with a subject exhibiting one or more features characteristic of a susceptibility to, or risk of developing, a disease, disorder, or (abnormal) condition. A particular subject is a patient. In another aspect, the subject is an individual for whom a diagnosis has been established and/or who has been exposed to a therapeutic treatment or who has been administered with a therapeutic compound/agent.

In a particular aspect, the subject is a human, in particular a child, an infant, an adolescent or an adult, in particular an adult of at least 18 years old, preferably an adult of at least 40 years old, still more preferably an adult of at least 50 years old. if the subject is a human subject, this “human subject” can be as also herein identified as an “individual”.

As used herein, the term “biological sample” typically refers to a sample obtained or derived from a biological source (e.g., a tissue, organism or cell culture) of interest, as described herein. In a particular aspect, the biological source is or includes an organism, such as an animal or a human. The biological sample may include a biological tissue or fluid. The biological sample can be or include cells, tissue, or bodily fluid. The biological sample can consist of, or include, blood, blood cells, free floating nucleic acids such as DNA, a biopsy sample, ascites, surgical specimen, cell-containing body fluid, sputum, saliva, feces, urine, cerebrospinal fluid, peritoneal fluid, pleural fluid, lymph, gynecological fluid, synovial fluid, secretion, excretion, skin swab, vaginal swab, oral swab, nasal swab, washing or lavage such as a ductal lavage or bronchioalveolar lavage, aspirate, scraping, and/or bone marrow.

In a particular aspect, the biological sample consists of, or include, samples obtained from a single subject or from a plurality of subjects.

In an embodiment, the biological sample is a biopsy, particularly a solid or liquid biopsy.

Tissue biopsies require solid matter from the subject's body. This biopsy is generally removed from a solid tumor or from tissues or organs suspecting to comprise tumor cells. Tissue biopsies are generally utilized when a known tumor's location is suspected or confirmed and available for extraction.

The biological sample is preferably a liquid biopsy. The liquid biopsy sample is for example a blood, plasma, serum, sputum, bronchial fluid or pleural effusion sample.

The biological sample is preferably derived from blood, e.g. blood serum (also herein identified as “serum”) or blood plasma (also herein identified as “plasma”), preferably plasma.

In a preferred embodiment, the DNA sequence of interest, typically the cfDNA sequence of interest, is obtain by patient blood collection followed by plasma isolation and DNA extraction from plasma.

Various methods of isolating nucleic acids from a sample (e.g., of isolating cfDNA from blood or plasma) are known in the art. Nucleic acids can be isolated, e.g., without limitation, with a standard DNA purification technique, for example by direct gene capture (e.g., by clarification of a sample to remove assay-inhibiting agents and capturing a target nucleic acid, if present, from the clarified sample with a capture agent to produce a capture complex, and isolating the capture complex to recover the target nucleic acid).

In a particular aspect, the subject is a human subject diagnosed or seeking diagnosis as having, diagnosed as or seeking diagnosis as at risk of having, and/or diagnosed as or seeking diagnosis as at immediate risk of having a cancer.

As used herein, the terms “cancer,” “malignancy,” “neoplasm,” “tumor,” and “carcinoma,” are used interchangeably to refer to a disease, disorder, or condition in which cells exhibit, or exhibit relatively, abnormal, uncontrolled, and/or autonomous growth, so that they display or displayed an abnormally elevated proliferation rate and/or aberrant growth phenotype. In a particular aspect, the cancer includes one or more tumors. In a particular aspect, the cancer consists of, or include, cells that are precancerous (e.g., benign), malignant, pre-metastatic, metastatic, and/or non-metastatic. In a particular aspect, the cancer consists of, or include, a solid tumor. In a particular aspect, the cancer consists of, or include, a hematologic tumor. In the context of the present invention, the cancer may be, for example, a colorectal cancer, hematopoietic cancer such as a leukemia, lymphoma (Hodgkin's and non-Hodgkin's), myeloma or myeloproliferative disorder, for example a sarcoma, melanoma, adenoma, carcinoma of solid tissue, in particular a squamous cell carcinoma of the mouth, throat, larynx, and/or lung, a liver/bile duct cancer, a genitourinary cancer such as a prostate, cervical, bladder, urothelial tract, ovary, uterine, and/or endometrial cancer, a renal cell carcinoma, bone cancer, pancreatic cancer, skin cancer, cutaneous cancer, intraocular melanoma, uveal melanoma, cancer of the endocrine system, cancer of the thyroid gland, cancer of the parathyroid gland, head and neck cancer, breast cancer, gastro-intestinal cancer, nervous system cancer.

In another aspect, the subject has a benign tumor/lesion such as for example a papilloma-induced tumor/lesion.

Preferably the cancer to be detected with a method or kit according to the invention is selected from the group consisting of colon cancer, breast cancer, lung cancer, uveal melanoma, ovary cancer and stomach cancer.

In a particular embodiment, the cancer to be detected with a method or kit according to the invention is an early-stage cancer, in particular a cancer of stage I, II or III.

As used herein, the term “stage of cancer” refers to a qualitative or quantitative assessment of the level of advancement of a cancer. In a particular aspect, the criteria used to determine the stage of a cancer includes, for example, one or more of the following: localization of the cancer in a body, tumor size, whether the cancer has spread to lymph nodes, whether the cancer has spread to one or more different parts of the body, etc. In a particular aspect, the cancer is cancer which has been staged using the so-called TNM System, according to which T refers to the size and extent of the main tumor, usually called the primary tumor; N refers to the number of nearby lymph nodes that have cancer; and M refers to whether the cancer has metastasized. An early-stage cancer is a term used to describe cancer that is early in its growth, and may not have spread to other parts of the body. It particularly refers to stage I, II and optionally III. In a particular aspect, the cancer is a Stage I-III (cancer is present; the higher the number, the larger the tumor and the more it has spread into nearby tissues), or Stage IV (the cancer has spread to distant parts of the body) cancer. In a particular aspect, the cancer is assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); or unknown (there is not enough information to identify cancer stage).

### Applications

Methods and kits of the present invention can be used in a variety of applications. For example, methods and kits of the present disclosure can be used to screen, detect or diagnose, or aid in screening for, a cancer.

In a particular aspect, screening uses a method and/or a kit as disclosed herein, and will provide the diagnosis of a condition, e.g., a type, origin or stage of cancer.

Those of skill in the art will appreciate that regular, preventative, and/or prophylactic screening for a cancer improves diagnosis and treatment efficiency if needed. As noted above, early-stage cancers include, according to at least one system of cancer staging, Stages I to Ill of cancer. Thus, the present disclosure provides methods and kits particularly useful for the early diagnosis and treatment of cancer.

In a particular aspect, the cancer screening in accordance with the present invention is performed once or multiple times for a given subject. In a particular aspect, the cancer screening is performed on a regular basis, e.g., every six months, annually, every two years, every three years, every four years, every five years, or every ten years.

The method according to the invention can be carried out in a subject which is asymptomatic at the time of screening, so that methods and kits of the present invention are especially likely to detect early-stage cancer.

In a particular aspect, the screening using a method and/or a kit of the present disclosure can be followed by a further diagnosis-confirmatory assay, which further assay can be performed to confirm a diagnosis resulting from a method as disclosed herein, such as a method involving a solid biopsy or imagery.

Any of the herein described method can be used in order to decide on the initiation of a therapy or to select an appropriate/optimized therapy in a subject, in the presence of a tumor.

In a particular aspect, the invention also relates to a method for treating a tumor, which method may optionally comprise the performance of a diagnostic method according to the invention, and wherein the tumor is treated if present, for example if the subject has been identified as a cancerous subject.

The invention also relates to the administration of a suitable anti-cancer treatment, in particular of a drug/medicament or combination of drugs/medicaments, optionally together with surgery and/or radiotherapy, when a method according to the invention shows that the subject suffers from cancer.

Alternatively, the diagnostic method can be used in order to carry out an additional diagnostic step in the event of detection of a tumor, resulting for example from the analyze of a solid biopsy and/or from a tumor image.

### BRIEF DESCRIPTION OF THE FIGURES

FIG. 1. Targeting DNA-methylation patterns of primate-specific LINE-1 elements from plasma DNA. A. CpG density along the structure of a human specific LINE-1 (L1HS) element which contains 95 CpG, the L1PA_cfDNAme assay targets 34 CpG (about 30%). Each target amplicon is highlighted by a black bar below the structure. The number of CpG sites detected per amplicon is displayed in blue. B-C. Circos plots displaying the hits obtained across the genome with the L1PA_cfDNAme assay. B. This panel displays the overlapping hits obtained in the healthy donor plasma versus an ovarian cancer tissue, both deep sequenced. C. This panel displays the overlapping hits obtained in the healthy donor plasma versus a uveal melanoma tissue, both deep sequenced. D. Histogram summarizing the most represented sub-families of L1 targeted by the L1PA_cfDNAme assay in the 3 deep sequenced samples. Each bar corresponds to a sample, in the following order: Healthy=healthy plasma, 54M reads; OVC=ovarian cancer tissue, 44M reads; UVM=uveal melanoma tissue, 46M reads. L1 sub-families are ordered by most represented (sum of copies targeted by the assay across the 3 deep sequenced samples used, descending order). The colors highlight the relative contribution of L1PA copies hit by reads uniquely mapped in black, reads randomly mapped in grey, and by both in cross-hatched). The L1 copies are obtained by finding the overlap between all the hits, and annotated copies of L1P/L1HS in repeatmasker (hg38 genome version). Each copy is counted once.

FIG. 2. L1PA hypomethylation is detectable form plasma DNA in multiple forms of cancer. A. Average levels of methylation in healthy plasma, healthy tissues collected next to ovarian tumors, ovarian tumors and breast tumors. The average level of methylation for each sample, corresponds to the percentage of CG dinucleotides at each CpG site averaged by the number of CpG sites. The p-values are computed using Student's t-test (p_{HP}=0.026, p_{OvcTumors}=1.5e-05, p_{BrcTumors}=1.4e-05). B. Average levels of methylation in 6 different types of cancer, including 4 metastatic stages cohorts and 3 non-metastatic stages cohorts. The average level of methylation for each sample corresponds to the percentage of CG dinucleotides at each CpG site averaged by the number of CpG sites. The p-values are computed using Student's t-test (pcolon_M+<1e-4, pbreast_M+<1e-4, plung_M+=0.0377, puvea_M+=0.0017, povary_M0<1e-4, pstomach_M0<1e-4, pbreast_M0=0.0227). Black dotted lines represent the median. C. Methylation level at each targeted CpG sites (x-axis) for each sample (y-axis), depicted as a heatmap. No clustering is done on the data, which comes ordered by targeted CpG site on the x-axis (amplicon # are indicated), and sample type (healthy donor, colorectal cancer, breast cancer or ovarian cancer plasmas) on the y-axis. The metaplots represent the average levels for healthy versus cancer samples at each CpG site. D. Differential methylation levels between healthy samples and patients for each type of cancer. CG sites are ordered from 5′ to 3′ along the L1 structure. P-values were computed using Student's t-test (corrected for multiple tests using R function p.adjust, method “BH” corresponding to Benjamini & Hochberg (1995)).

FIG. 3. L1PA methylation changes discriminate cancer samples from healthy donors. A-B. Receiver Operating Characteristic (ROC) curves obtained for plasma samples classification using proportion of methylation at the 33 CpG targeted in all cancer samples (A) or by cancer subtypes (B). C-D. ROC curves obtained for plasma samples classification using the proportion of haplotypes within each amplicon targets in all cancer samples (C) or by cancer subtypes (D). All classifications include 5000 stratified random repetitions of learning on 60% of the samples and testing on the 40% left with bootstrapping. CRC_M+n=75, BRC_M+n=97, NSCLC_M+n=52, UVM n=70, OVC_M0 n=23, GAC_M0 n=27, BRC_M0 n=40 tested versus 123 healthy donors. E. Sensitivity at 99% specificity by cancer class in the two models (ordered by increasing sensitivity, bars indicate 95% Cl).

FIG. 4. Cancer sample identification performances are reproducible on an independent cohort.

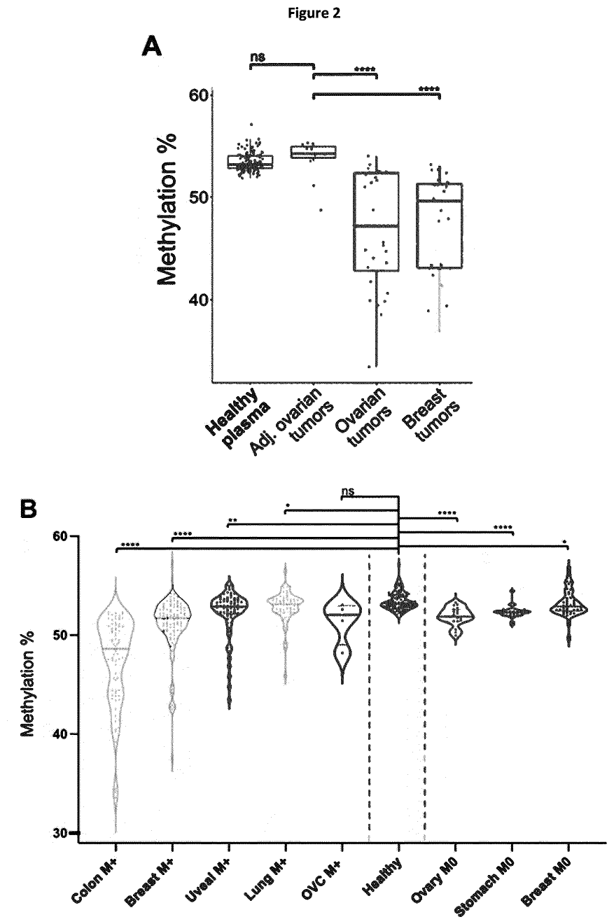

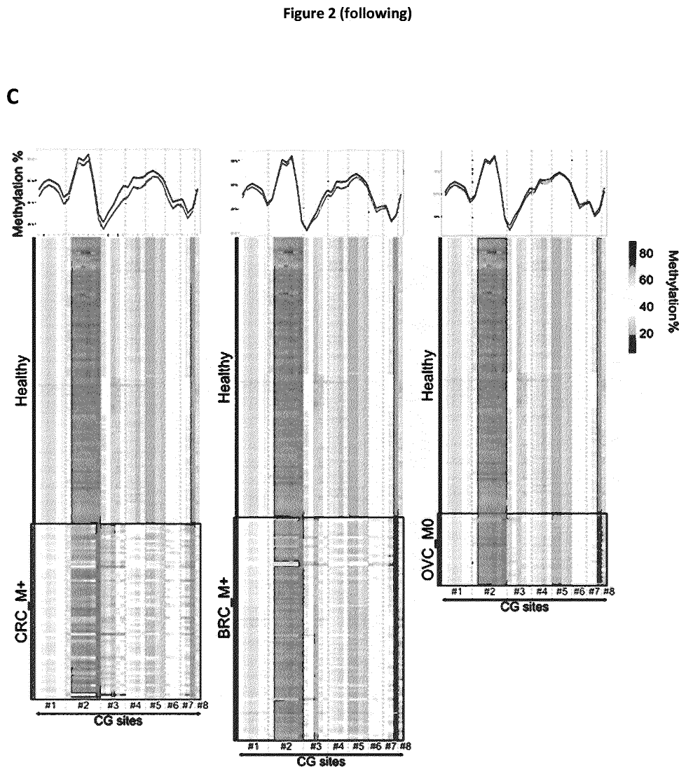

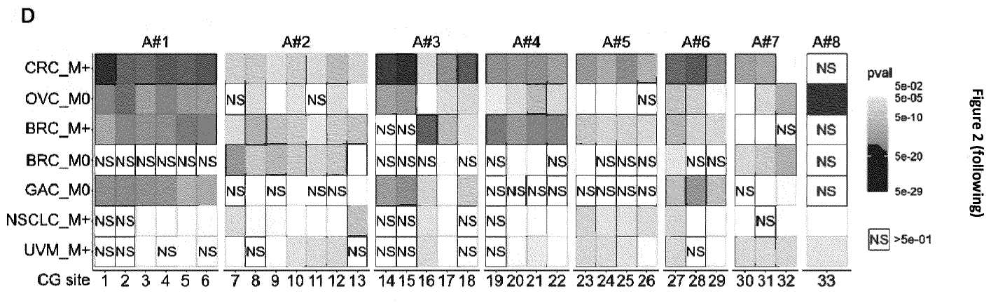

A. Table displaying the number of patients for each cancer type and stages (non-metastatic: M0 vs metastatic: M+) in cohort 1 and cohort 2. B. Methylation level at each targeted CpG sites (x-axis), for each healthy sample (y-axis) form cohort 1 vs cohort 2, depicted as a heatmap. No clustering is done on the data, which comes ordered by targeted CpG site on the x-axis (amplicon # are indicated). The metaplots represent the average levels for donors of cohort 1 versus cohort 2 at each CpG site (grey intensity correspond to the legend highlighted on the side of the heatmap). C. Comparison of the average levels of methylation, excluding amplicon 3, in cohorts 1 and 2 in healthy donors and the 5 different types of cancers in common in the two cohorts. Methylation levels are calculated as explained previously in FIG. 2. The p-values are computed using Student's t-test (p_{ovary_M0_C1vsC2}<1e-4, p_{stomach_C1M0vsC2M+}=2e-4). Doted lines represent the median. D-E. ROC curve obtained with the classifier trained on cohort 1 and tested on cohort 2 using the proportion of methylation at each CpG target, except CpGs of amplicon 3 in all cancer samples jointly (D) or by cancer types (E). No bootstrapping steps. F. Sensitivity at 99% specificity by cancer class using classifiers based on single-CpG methylation levels, excluding (dark grey) or including (light grey) amplicon 3. Bars indicate 95% Cl.

FIG. 5. The origin of the cancer can directly be inferred from the L1PA methylation status

A-D. ROC curves obtained with the ‘Multiclass’ classifier, trained on cohort 1 and tested on cohort 2 using single CpG methylation levels with (A) or without amplicon #3 and bootstrapping (B) or using the proportion of haplotypes with (C) or without amplicon #3 and bootstrapping (D). Only classes which were homogeneous between the cohort 1 and 2 were included in this test, i.e. healthy donors, CRC_M+, BRC_M+ and OVC_M0M+. E. AUC by cancer class in the two types of models (methylation a single CpGs or haplotype proportions) with amplicon #3 and bootstrapping or without amplicon #3 and without bootstrapping. Bars indicate 95% Cl.

FIG. 6. Scheme of the targeted bisulfite sequencing strategy used to build the L1PA-cfDNAme libraries. The protocol starts by the incorporation of unique molecular identifiers (UMI) via 1 cycle of linear PCR in order to identify each initial molecule present in the sample. Inventors also incorporated a 2^{nd} set of molecular identifiers (UID) during the 2^{nd} PCR in order to generate libraries with enough nucleotides diversity which is crucial for a successful downstream sequencing (See method section for more details).

FIG. 7. Summary flow chart illustrating the pipeline developed for reference-free alignment of the sequencing data.

FIG. 8. A. Cancer detection rates with L1PA_cfDNAme vs common recurrent mutations assessed in previous studies. B. Cancer detection rates with L1PA_cfDNAme vs the non-invasive Galleri test developed by Grail.

FIG. 9. ROC curve obtained with the classifier trained on cohort 1 and tested on cohort 2 using the proportion of haplotypes, except CpGs of amplicon #3.

### EXAMPLES

The following examples are presented for illustrative and non-limiting purposes and serve to illustrate the invention.

### Materials & Methods

### Methods

### Preparation of Plasma DNA

Whole blood was collected in BD Vacutainer EDTA tubes (BD Biosciences). Plasma was isolated within 3 hours from blood draw to ensure good quality cell free DNA. Blood was centrifuged at 820 g for 10 min at room temperature. Subsequently, the supernatant was transferred to a new 2 ml tube and centrifuged at 16 000 g for 10 min at 15° C. to remove the remaining cellular debris. The plasma was collected and transferred into new 2 ml tubes, which were stored at −80° C. for further processing. DNA was extracted from 2 ml of plasma using the automated QIAsymphony Circulating DNA kit (Qiagen) or manual QIAamp circulating nucleic acid kit (Qiagen), according to the manufacturer's instructions, and isolated DNA was eluted in 60 μl or 36 μl of elution buffer, respectively. Plasma DNA was quantified by Qubit® 2.0 Fluorometer using Qubit® dsDNA HS Assay Kit (Thermo Fisher Scientific) according to the manufacturer's instructions and stored at −20° C. until use.

Preparation of DNA from Cell Lines and Tissues

Isolation of DNA from cell lines and peripheral blood mononucleated cells (PBMC) was performed using the QIAamp DNA Mini Kit or QIAamp DNA Blood Mini Kit (Qiagen) according to the manufacturer's instructions. DNA from cryopreserved and formalin-fixed paraffin embedded (FFPE) tumor tissues was extracted using a classical phenol chloroform protocol and the NucleoSpin® FFPE DNA kit (Macherey Nagel), respectively. Isolated DNA was quantified by Qubit® 2.0 Fluorometer using Qubit® dsDNA BR Assay Kit.

### Bisulfite Conversion

Bisulfite treatment of the isolated genomic DNA from the cancer tissues, cancer cell lines and PBMC was performed using an EZ DNA Methylation-Gold Kit™ (Zymo Research, CA, USA), following the manufacturer's instructions. Up to 200 ng of genomic DNA was treated with Zymo CT Conversion Reagent for 10 min at 98° C. in a thermal cycler and then for 2.5 hours at 64° C. Bisulfite-treated DNA was purified via spin columns supplied in the kit. Bisulfite treatment of plasma DNA was performed using the Zymo EZ DNA Methylation-Lightning Kit™ (Zymo Research, CA, USA), according to the manufacturer's instructions.

DNA isolated from 2 ml of plasma (up to 200 ng) was treated with Zymo Lightning Conversion Reagent with the following cycling conditions: 98° C. for 8 min and 54° C. for 60 min. Bisulfite-treated DNA was purified via spin columns supplied in the kit. Bisulfite-treated DNA was stored at −70° C. and further used to build a sequencing library.

### Primer Design

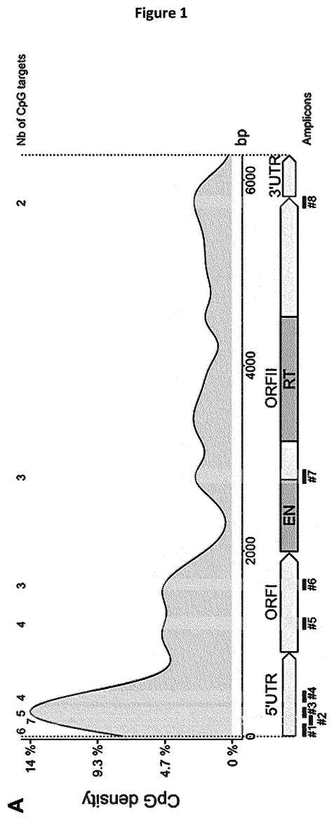

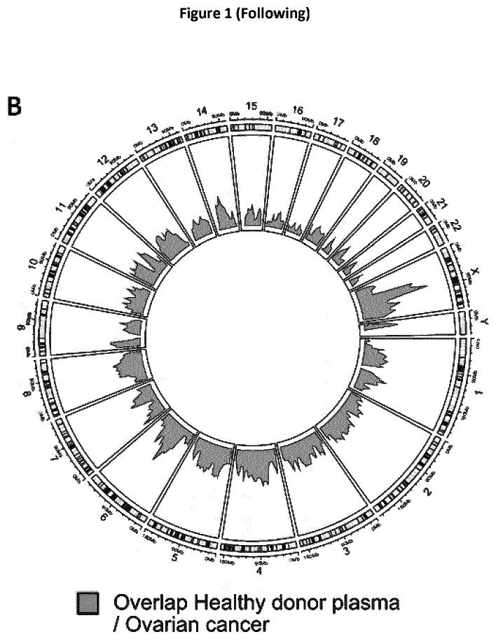

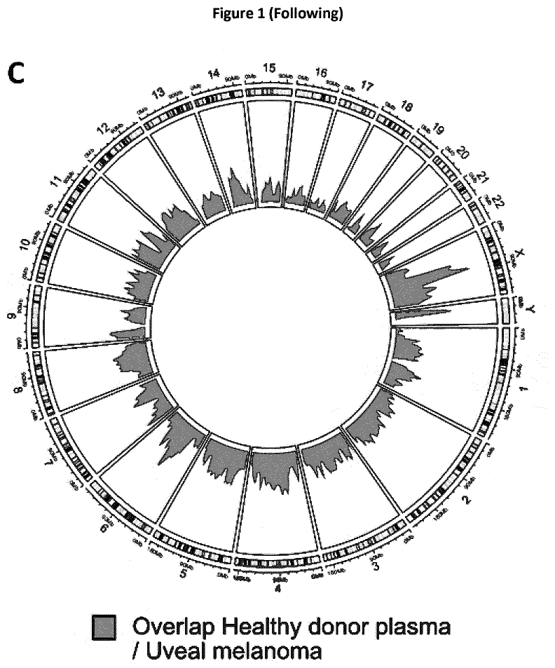

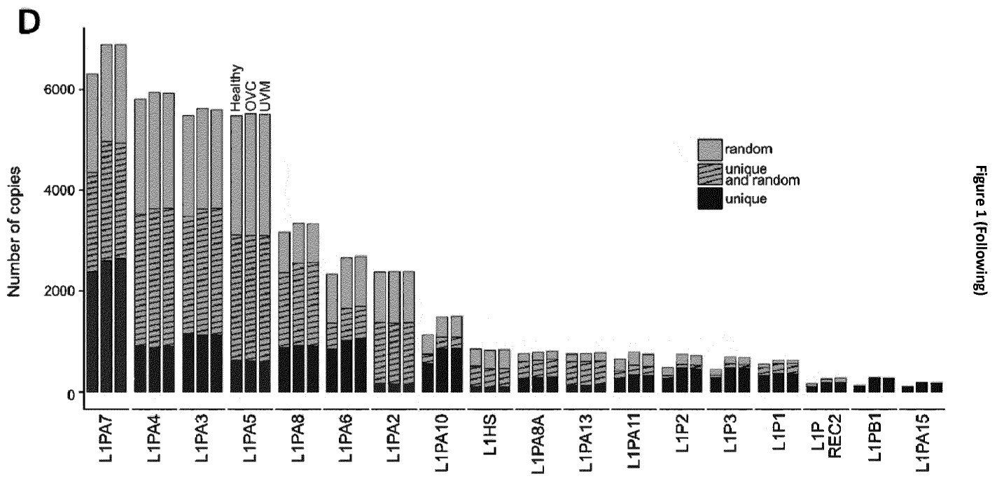

Eight primer pairs were designed using the LINE-1 Human Specific consensus sequence from Repbase (FIG. 1A). Although 5′UTR (promoter region) is CpG-rich and common target for methylation quantitation, L1PA copies are frequently 5′-truncated. Therefore, primers were also designed for ORFI and ORFII and 3′UTR to target more L1PA elements and improve the sensitivity of the assay. All primers were designed for plus strand of bisulfite converted DNA, using the MethPrimer or PyroMark Softwares. Targeted regions ranged from 101 bp to 150 bp, to better capture plasma DNA fragments which have a mean size of 170 bp and contained 2-7 CpG targets. Primers were methylation-independent with 0 to 2 CpG sites included and none toward the 5′ end of the primers. In order to avoid methylation-biased amplification, degenerated primers were used, targeting both of the methylated and unmethylated states for primers including CpG sites. The target-specific primers both contained Fluidigm universal CS (common sequence) tags at their 5′ ends for a later amplification step. A 16 N (random nucleotide sequences) was incorporated as a unique molecular identifier (UMI) sequence between the target-specific sequence and the CS2 in the reverse primers to allow for the identification of unique individual molecule and accurate scoring of DNA methylation rates. These primers can generate 4294 million distinct UMIs. As LINE-1 covers thousands of copies per genome, a high number of distinct UMIs was used for unique barcoding of each target molecule. One strand of each template molecule was encoded with a UMI using one cycle of linear target-specific PCR.

A 16 N stretch was incorporated between the target-specific sequence and the CS1 in forward primers to increase diversity of targeted sequencing libraries and improve sequencing quality. All primers were obtained from Eurogentec (RP-cartridge purification method). Seven amplicons (#1, #3, #4, #5, #6, #7, #8) were multiplexed in a single reaction. Amplicon #2 was processed individually as it overlaps with other primers. Designed primers were evaluated by in silico PCR using converted human genomic DNA as a reference.

### Preparation of Targeted Bisulfite Sequencing Libraries (FIG. 6)

Sequencing libraries were prepared using three PCR steps: target-specific linear amplification (UMI assignment), target-specific exponential amplification, and barcoding PCR.

Each library was prepared in two individual reactions (due to the overlap between primers), including:
1. Multiplex PCR amplification of 7 probes (L1HS-2, 4, 6, 7, 8, 9, 15), using Platinum™ Multiplex PCR Master Mix, Thermofisher, Life Technologies SAS
2. Single PCR amplification of one probe (L1HS-14), using Hot Star Taq Plus DNA Polymerase, Qiagen

For multiplex reaction, each primer was used at a final concentration of 0.01 to 0.06 μM (low concentration was used to avoid primer dimers), whereas for single reaction 0.1 μM primer was used. Up to 5 ng and 4 ng of bisulfite converted DNA (DNA from plasma and tissues) were used for multiplex and single reaction, respectively.

UMI assignment for multiplex reaction was performed using Platinum™ Multiplex PCR kit Master Mix (Thermofisher, Life Technologies SAS) in a 25 μL reaction containing 1x Platinum™ Multiplex PCR Master Mix, 0.01-0.06 μM mix of L1HS reverse primers (containing 16N) and up to 5 ng bisulfite converted DNA at the following thermocycling conditions: 95° C. for 5 min followed by 1 cycle at 95° C. for 30 s, 58° C. for 90 s, 72° C. for 40 s. UMI assignment for single reaction was performed using Hot Star Taq Plus DNA Polymerase (Qiagen) in a 25 μL reaction containing 1×Taq PCR Buffer, 0.65 U Hot Star Taq (5 U/L), 0.2 M dNTPs, 1.5 mM MgCl2, 0.1 μM L1HS-14 reverse primer (containing 16N), up to 4ng bisulfite converted DNA at the following thermocycling conditions: 95° C. for 10 min followed by 1 cycle at 94° C. for 60 s, 58° C. for 30 s, 72° C. for 40 seconds.

To ensure complete removal of the reverse primers and dNTPs, each 25 μL reaction was treated with 50 U of Exonuclease I (Thermo Fisher Scientific) and 10 U of FastAP Thermosensitive Alkaline Phosphatase (Thermo Fisher Scientific) at 37° C. for 1 h. Afterwards a heat inactivation at 80° C. for 15 min was done.

Target-specific exponential amplification for multiple reaction was performed using Platinum™ Multiplex PCR kit Master Mix in a 50 μL reaction containing 1× Platinum™ Multiplex PCR Master Mix, 0.01-0.06 μM mix of L1HS forward primers (containing 16N), 0.2 μM CS2 reverse primer and 20 μL of purified PCR product at the following thermocycling conditions: 95° C. for 5 min followed by 28 cycles at 95° C. for 30 s, 58° C. for 90 s, 72° C. for 30 s followed by a 10 min incubation at 72° C. Target-specific exponential amplification for single reaction was performed using Hot Star Taq Plus DNA Polymerase in a 50 μL reaction containing 1×Taq PCR Buffer, 0.65 U Hot Star Taq (5 U/μL), 0.2 μM dNTPs, 1.5 mM MgC2, 0.2 μM L1HS-14 forward primer (containing 16N), 0.2 μM CS2 reverse primer and 16 μl of purified PCR product at the following thermocycling conditions: 95° C. for 10 min followed by 25 cycles at 94° C. for 60 s, 58° C. for 30 s, 72° C. for 30 s followed by a 10 min incubation at 72° C.

PCR products of multiplex and single reaction were pooled together after quantification by qPCR. Pooled product was purified using Agencourt AMPure XP (Beckman Coulter) at 1.2× ratio according to the manufacturer's protocol. Purified DNA was eluted in 30 μl of water.

Barcoding PCR was performed using universal fluidigm primers to introduce sample-specific barcodes and complete sequencing adaptors. 25 μL of purified pooled PCR product, 1× Phusion HF Buffer, 1 U Phusion Hot Start II DNA Polymerase (Thermo Fisher Scientific), 0.2 μM fluidigm primer, and 0.2 mM dNTPs were mixed in the final volume of 50 μL and amplified with the following thermocycling conditions: 98° C. for 2 min, followed by 20-25 cycles of 98° C. for 10 s, 62° C. for 30 s, and 72° C. for 30 s followed by a 5 min incubation at 72° C.

Finally, amplified product was purified through double (upper and lower) size selection by two consecutive AMPure XP steps. At first step, a low concentration of AMPure XP beads (0.6×-0.7× ratio) was used. In this step, the beads containing the larger fragments are discarded and supernatant was collected (reverse purification) for the next step. At second step, more beads (1.1×-1.2× ratio) were used. In this step the beads containing the desired fragments were collected and purified according to the manufacturer's protocol. Size-selected libraries were eluted in 15 μL of low-EDTA TE buffer.

The libraries were quantified with fluorometric assay using Qubit HS DNA kit (Thermo Fisher Scientific). Afterwards the libraries were quantified and qualified with electrophoretic assay using Caliper LabChip HS DNA (PerkinElmer) or BioAnalyzer HS DNA kit (Agilent), and pooled equimolarly for sequencing. Sequencing was performed on Illumina HiSeq—rapid run mode or NovaSeq (PE30,170).

### Preprocessing of the Reads

The sequencing facility delivers a number of files in the FASTQ format. Each sample is a separate FASTQ file containing its corresponding raw sequences, composed by a number of sequences parts: CS1, forward UMI, forward primer, insert, reverse primer, reverse UMI, and CS2. For each sample, the reads are demultiplexed (i.e., cut, using program atropos) using forward and reverse primer sequences, in order to create per primer-set FASTA files containing inserts sequences and reverse UMIs for deduplication—the reverse UMIs being unique per input DNA molecule.

Inserts sequences and reverse UMIs are then filtered by expected sizes with a tolerance of preferably ±5 bases for the inserts, and no tolerance for the UMIs which are for example composed of at least 16 bases. Inserts and reverse UMIs sequences are then concatenated to form singular sequences, which are in turn deduplicated using for example program vsearch. Reverse UMIs are then trimmed, and inserts are isolated in separate FASTA files. On a primer-set basis, all resulting inserts for all samples are aggregated into a single FASTA file, resulting in one file per primer-set.

Clustering, extraction of reference sequences and global alignment (FIG. 7) Using for example program vsearch (preferably with the following parameters: --cluster_fast <inputFasta> --notrunclabels --fasta_width 0 --iddef 4 --id 0 --qmask none --clusterout_sort --consout <referenceFasta>), a clustering with minimum sequence identity is applied to each file (the clustering of the DNA subsequences is performed with the help of vsearch). The subsequence is added to the cluster if the pairwise identity with the centroid is higher than 0. The pairwise identity is defined as the number of (matching columns)/(alignment length), or a subsample of 20 million reads randomly chosen if a given file comprises more. Using for example program awk, the “n”, for example 10, largest clusters' reference sequences are isolated in separate files: one FASTA file per primer-set, each containing n (in the present example 10) reference sequences coming from the n largest clusters of sequences. Using for example program mafft (preferably with the following parameters: --textmatrix <custom_score_matrix.txt>--retree 2) on each of said files, the n (for example 10) reference sequences are aligned pairwise using a custom score matrix to favor dinucleotides CG/TG alignment over other possible dinucleotides combinations (e.g., AG/AC/TC . . . ), resulting in n (in this example, 10) reference sequences database FASTA files for each primer-set. Lastly, using for example program mothur [preferably with the following parameters: #align.seqs(candidate=<inputFasta>, template=<referenceFasta>, align=needleman, match=1, mismatch=−1, gapopen=−1, gapextend=0)] on each primer-set FASTA file, all sequences from all samples are aligned to the corresponding reference sequences database.

CG Calling To call CG dinucleotides sites of interest, a sliding window of 2 bp was used on all aligned sequences to determine the distribution of dinucleotides along each sub-sequences of the DNA sequence of interest (here, each amplicon target). The proportion of dinucleotides CG is computed at each location (sub-sequence of interest) along the sequence, as well as that of any other dinucleotides. A first threshold (for example ≥20%) of CG/TG dinucleotides proportion is used to determine whether the considered location (sub-sequence of interest) in the sequence qualifies as a CG site or not. After bisulfite treatment, the information of what constitutes an actual TG dinucleotide, or a TG dinucleotide resulting from the conversion of an unmethylated CG is lost. To mitigate this, a second threshold (for example ≥95%) of TG proportion is preferably applied at these preselected sites as to potentially eliminate them from CG sites selection for being actual TG sites (e.g., not resulting from bisulfite conversion).

### Methylation Levels and Haplotypes Extraction

From the aligned sequences, using for example a custom python script, the patterns/profiles of methylation are extracted and compiled into either average levels of methylation at each identified CG dinucleotide site or proportions of methylation haplotypes (methylation state of successive CpG sites within each sub-sequence of interest, for example amplicon), for each sample.

### Classification Models Trained by Machine Learning

The resulting data (represented either as average levels of methylation per CG site or proportions of methylation haplotypes) is used to do supervised training of statistical models, in particular using the random forest (Breiman, L. Random Forests. Machine Learning 45, 5-32 (2001). https://doi.org/10.1023/A:1010933404324) classifier algorithm from Python package Scikit-Learn (Pedregosa et al., JMLR 12, pp. 2825-2830, 2011), with the following hyperparameters: n_estimators=300, criterion=‘gini’, max_depth=None, min_samples_split=2, min_samples_leaf=1, min_weight_fraction_leaf=0.0, max_features=‘sqrt’, max_leaf_nodes=None, min_impurity_decrease=0.0, bootstrap=True, oob_score=False, warm_start=False, class_weight=None, ccp_alpha=0.0, max_samples=None.

The rationale for choosing random forest over other learning methods is mainly three-fold: it is less prone to overfitting; it shows excellent performance even when the quantitative relationship between features and observations is biased in favor of the former, such as when using methylation haplotypes data representation (WIREs Data Mining Knowl Discov 2012, 2: 493-507 doi: 10.1002/widm.1072); random forests also inherently return measures of variable importance, which greatly facilitate the interpretability of model decisions.

It is also worthy of note that most classifier learning algorithms from Scikit-Learn were tested during the development of this test, and random forest was one of the top performers.
The features used to train the models are either the average levels of methylation per CG site (n=33), or the proportions of methylation haplotypes (i.e., the combinatorial of all the possible methylation status of CG sites within a given amplicon) (n=274). No additional transformation nor feature selection is performed on the data.
Models evaluation is done as follows: classifications are run 5000 times in order to estimate variance and confidence intervals. Each run, as many samples from each class are randomly drawn without replacement from the training data set. The samples from these draws are stratified by class and split into 60% for training, 40% for evaluation.

The true and false positive rates are evaluated at each run, with interpolation to generate all points of the ROC curve; at the end of the 5000 runs, an average ROC curve is generated and the 95% confidence interval is calculated based on the results of all runs. In the context of multiclass, the ROC curves of each class are generated by taking the class under consideration as the positive class (there is therefore no particular weight associated with the control class, i.e., the healthy plasmas).

### Results

### Targeting Primate-Specific LINE-1 Elements Informs on Plasma DNA-Methylation Patterns Genome-Wide (FIG. 1)

The inventors developed a method, in particular a PCR-based targeted bisulfite method coupled to computer-implemented sequencing (also herein identified as “deep sequencing”) to detect methylation patterns of DNA, in particular of circulating cell free DNA. They used sodium bisulfite-based chemical conversion to achieve base-pair resolution analysis, which is preferable to address methylation levels at single CpG dinucleotides and the co-methylation of multiple CpG sites to determine methylation haplotypes (methylation state of successive CpG sites). They have in particular designed 8 amplicons (target sub-sequences of interest) targeting primate specific L1 elements (L1PA) for use in multiplexed PCR (FIG. 1A). The primers were equipped with unique molecular identifiers (UMIs), which helps for signal deconvolution and the detection of true low frequency alterations, and for reducing errors. The inventors detected thousands of L1PA elements scattered throughout the genome as observed by the genomic hits obtained from a healthy plasma sequenced at high depth (data not shown). The inventors observed a very similar pattern for ovarian cancer and uveal melanoma tissues, also sequenced at high depth (FIG. 1B-C), as well as healthy and cancer plasma with standard coverages. This demonstrates the robustness of the approach. Overall, the estimated number of L1PA elements targeted is around 30-40,000 per genome including half of the human specific copies (L1HS) and many copies of the other L1PA subfamilies. This represents 82-125,000 CpG sites (FIG. 1D).

Following deep sequencing of multiplexed PCR, reads are traditionally mapped back to the genome. However, the vast majority (about 80%) of the sequencing reads from repetitive sequences are lost or assigned randomly during mapping steps and subsequently lost for classical differentially methylated region (DMR) calling. Thus, the inventors developed computational tools to optimize (increase accuracy/performance) the align repetitive sequencing data without using a reference genome. To perform this, they clustered all good quality reads obtained based on their similarity and extracted a reference sequence from each cluster. Next, they aligned all the reads onto the reference sequences identified, with a computational time complexity of O(n) (n being the number of reads to align and O being the Big O of Landau), allowing the alignment of millions of reads. This genome reference-free alignment, allows to extract the informative CpG sites agnostically. The inventors selected the sites (target sub-sequences of interest) with a minimum CG/TG content; in particular ≥20% including preferably at least 5% of CG, to increase the likelihood, preferably ensure, that the CG position or site of interest displays methylation. This selection was done on healthy samples to avoid biases related to cancer hypomethylation. The inventors retrieved 33 of the 34 CpG positions covered by the patient panel with respect to the L1HS consensus sequence. The 34^{th} CpG is not present in copies belonging to the L1PA6-8 families, which represent 32% of the hits obtained. This led to a CG/TG signal below the 20% threshold used for CpG site identification in the particular example.

Overall, the inventors implemented an unbiased method without relying on current genomic annotations, which remain poor for repeated sequences of interest, to retrieve methylated sites contained by the youngest LINE-1 elements present in the human genome. With the method disclosed herein, the inventors were able to study DNA-methylation, in particular cfDNA-methylation, levels and patterns, overall and at each CpG site.

L1PA Hypomethylation is Detectable from Plasma DNA in Multipleforms of Cancers (FIG. 2)

The inventors validated their method on cancer cell lines, healthy tissues and tumor tissues and observed a statistically significant L1PA hypomethylation in ovarian and breast tumors compared to healthy plasma samples and healthy tissues collected adjacent to ovarian tumors (FIG. 2A). Next, plasma samples from colorectal and ovarian cancers were tested, in which a substantial rate of L1 hypomethylation has previously been reported. The inventors detected a highly statistically significant L1PA hypomethylation in cfDNA of metastatic stages of colorectal cancers (CRC_M+) and stages Ill/IV of ovarian cancers (OVC_M0/M+ composed of 80% stage III). The inventors also detected a highly statistically significant L1PA hypomethylation in metastatic stages of breast cancer (BRC_M+) and uveal melanoma (UVM_M+) as well as early stage of gastric cancer (GAC_M0) (FIG. 2B). The hypomethylation was less significant in metastatic non-small cell lung cancers (NSCLC_M+) and early stage of breast cancer (BRC_M0). Indeed, focusing on global methylation levels provides only part of the information. The inventors further computed the levels of methylation at each CpG site of the target L1HS sequence (n=33). They observed specific patterns of methylation along the L1 structure through the various amplicons (i.e. sub-sequences of interest) (FIG. 2C). Overall, these patterns are robust among the 123 healthy plasma samples tested. As expected, the 5′ part of the L1 is heavily methylated, in particular within the 2^{nd} amplicon, but the last CG target, part of amplicon #8, is also heavily methylated (62% in healthy samples). The inventors observed that the most methylated sites are the 5 central CpGs of amplicon #2, which are flanked by two CpG sites carrying low methylation levels. However, this is not necessarily the sites presenting the most significantly different levels of methylation between healthy and cancer samples (FIG. 2D). The inventors observed that all CpG sites display differential patterns in one or more cancer subtypes and that different CGs can be informative in different groups. For example, the 2 first CpGs of amplicon #3 are highly significant in metastatic colorectal cancers (CRC_M+) but not in metastatic breast cancer (BRC_M+). This suggests that L1PA hypomethylation patterns vary in different types of cancer.

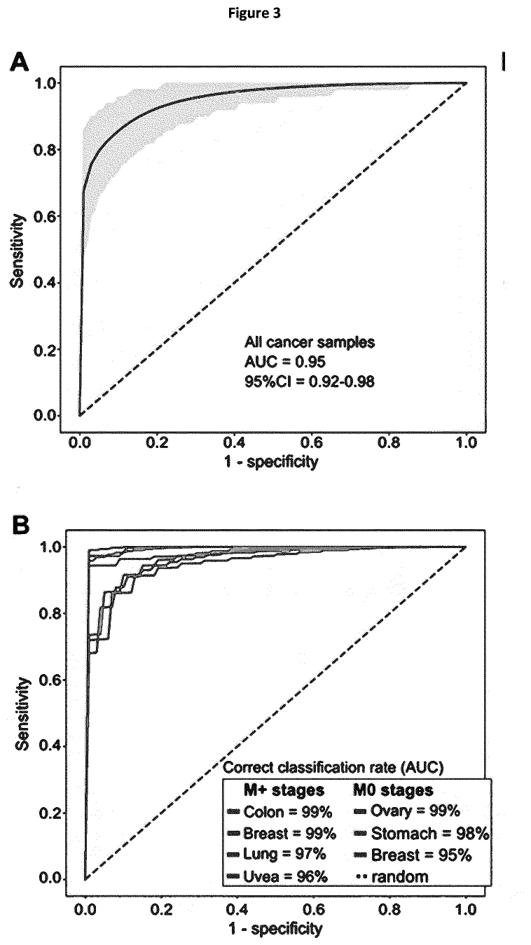

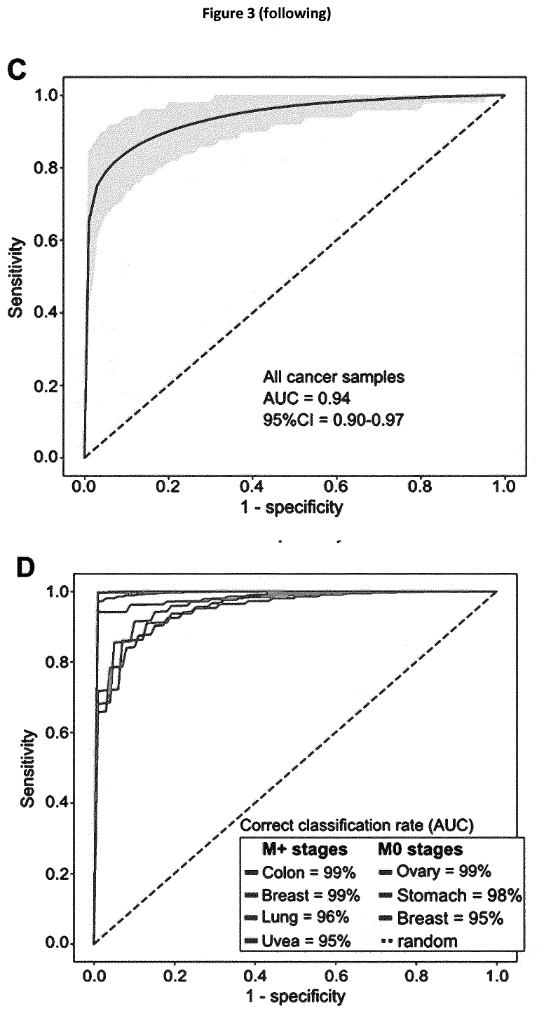

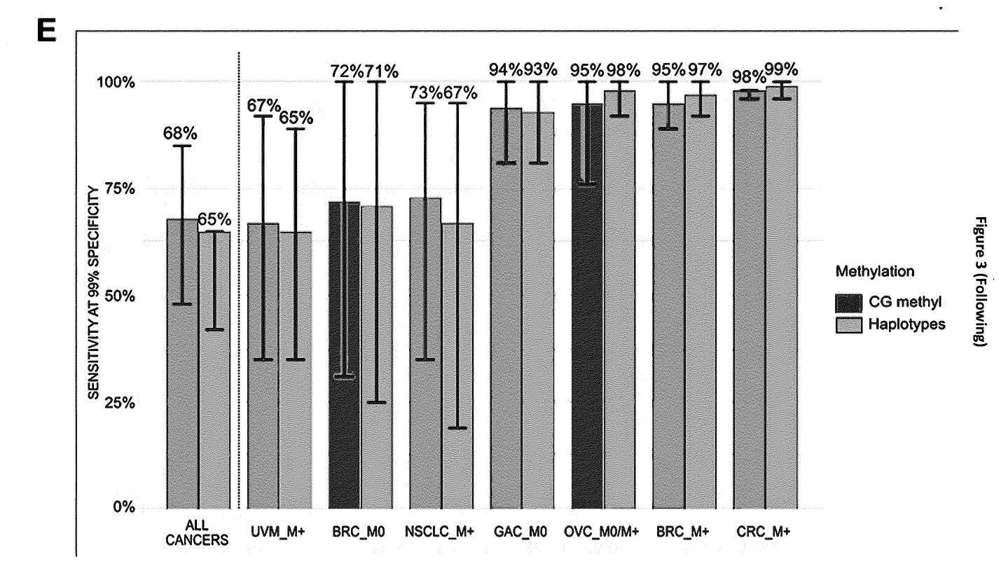

L1PA Hypomethylation-Based Classifiers Discriminate Cancer Samples from Healthy Donors in Multiple Forms of Cancers (FIG. 3)

The inventors trained a classification model, using a random forest algorithm based on these 33 CpG sites corresponding to the levels of methylation at each CpG target, to assess its classification potential in discriminating healthy from tumor plasma. The methylation of L1PA elements, showed an extremely performant ability to discriminate between healthy and tumor plasmas from 6 different types of cancers with an area under the curve (AUC) of 0.95 (95% confidence interval (CI)=0.92-0.98, FIG. 3A). The model is extremely performant for metastatic stages of colorectal and breast cancers (AUC_CRC_M+=0.99; 95% CI=0.99-1.00; AUC_BRC_M+=0.99; 95% CI=0.99-1.00) but also for stages III/IV of ovarian and non metastatic gastric cancer (AUC_OVC_M0=0.99; 95% CI=0.98-1.00; AUC_GAC_M0=0.98; 95% CI=0.93-1.00) (FIG. 3B). Additionally excellent performances are observed for metastatic lung cancers and uveal melanoma (AUC_NSCLC_M+=0.97; 95% CI=0.92-1.00; AUC_UVM_M+=0.96; 95% CI=0.92-0.99) and more importantly for early stages of breast cancer (AUC_BRC_M0=0.95; 95% CI=0.89-1.00).

The inventors also developed an approach integrating methylation haplotypes at the single molecule level. This corresponds to true patterns of methylation of adjacent CpGs detected for each molecule amplified. Based on the combination of the 33 targeted CpGs, the inventors were able to extract a total of 274 unique haplotypes from the 33 CpG sites. Here, the inventors wanted to see if the classification could be improved with a more detailed signal. They observed similar results than with the methylation levels at each CpG site (FIG. 3C-D). However, in the case of ovarian cancer, the model based on haplotypes is even more performant and robust (FIG. 3E).

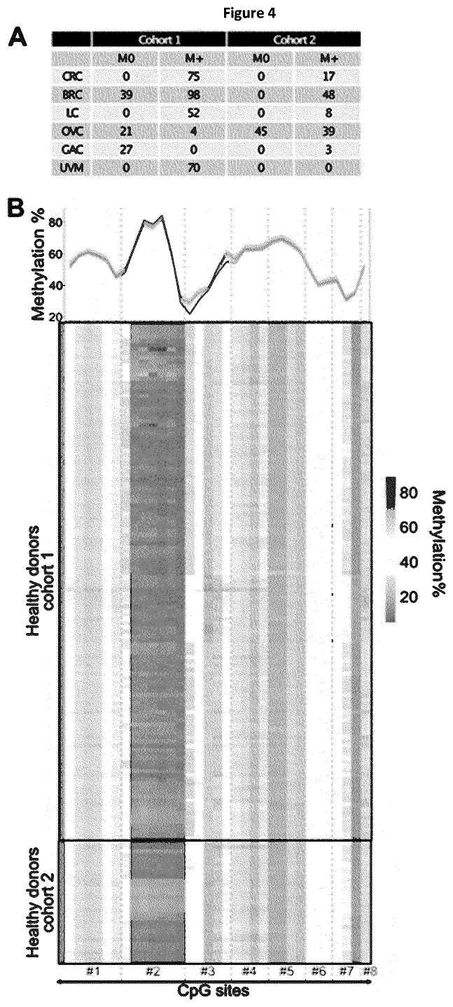

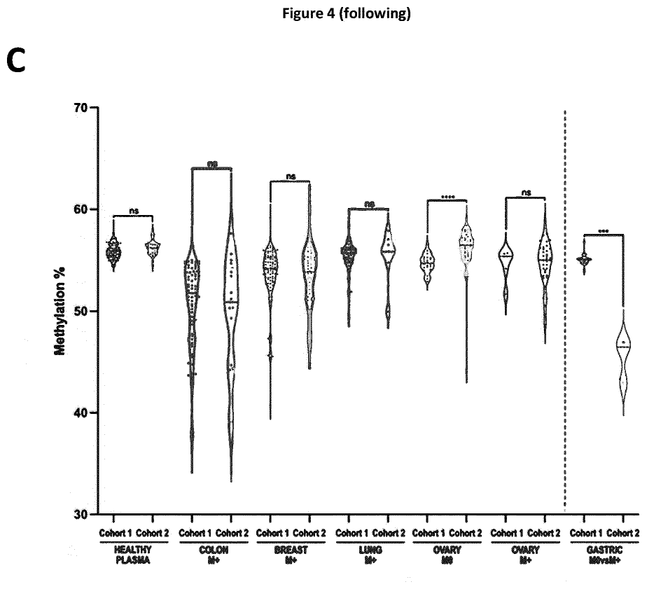

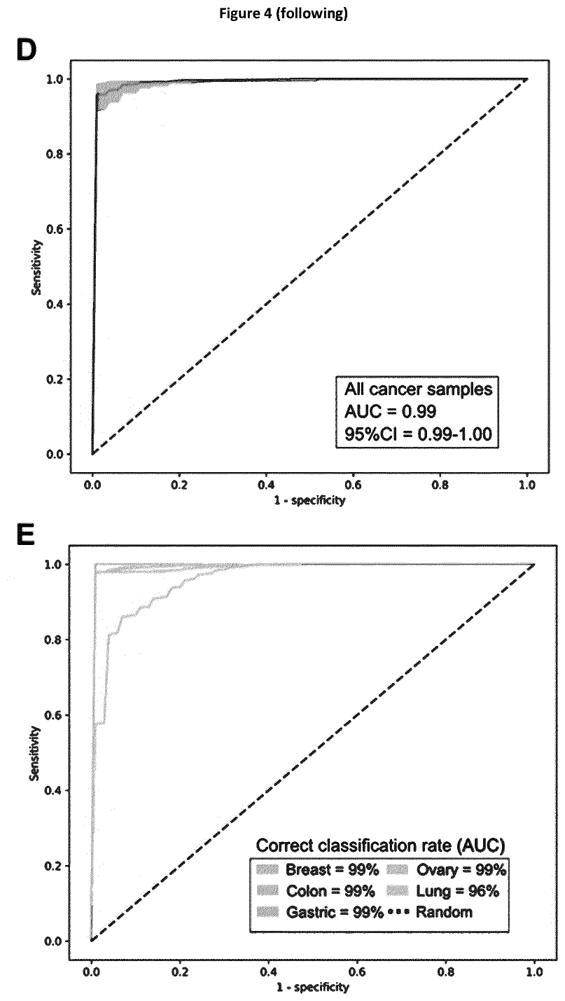

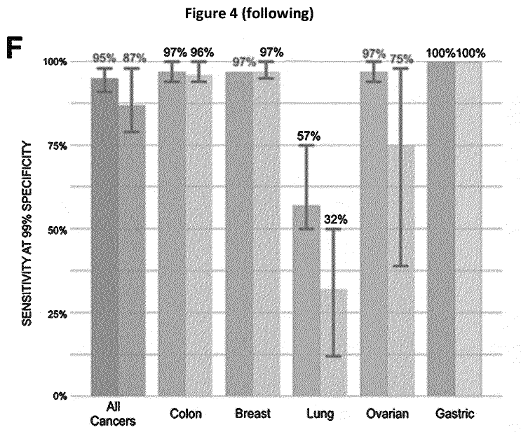

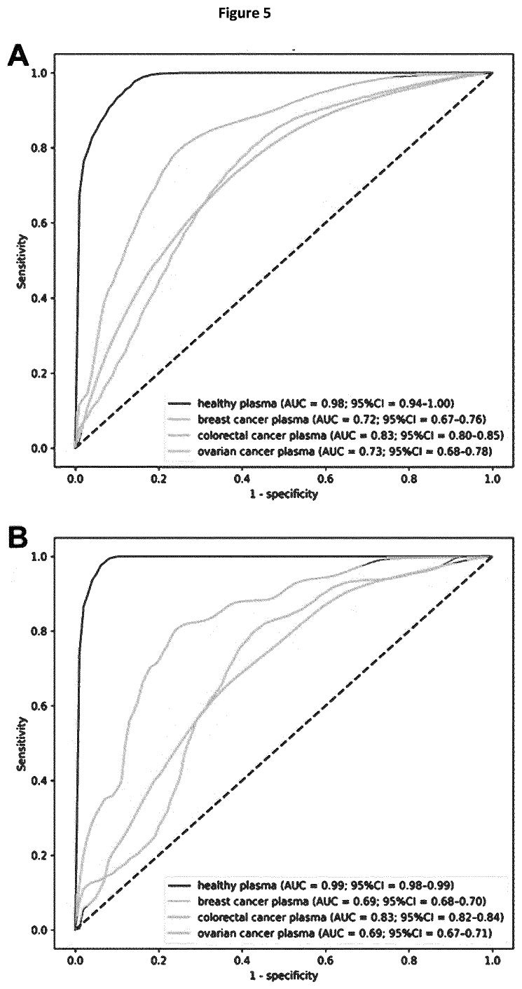

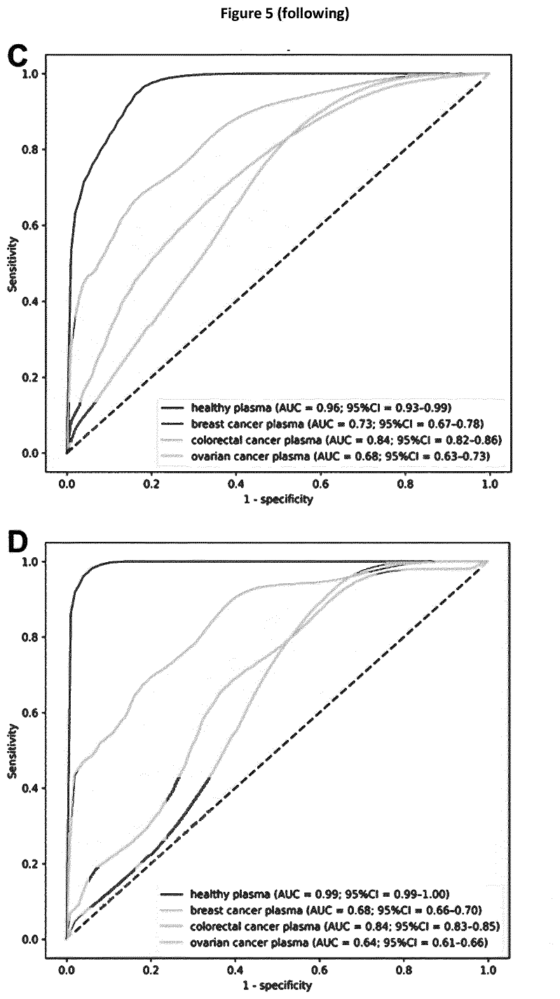

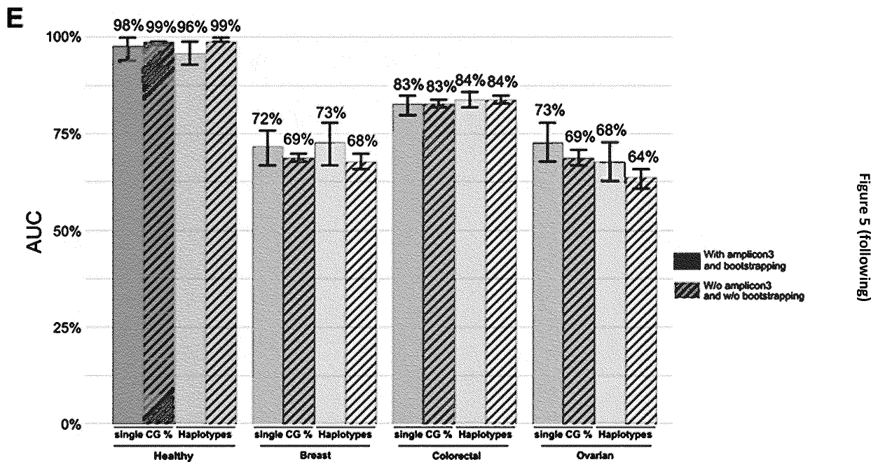

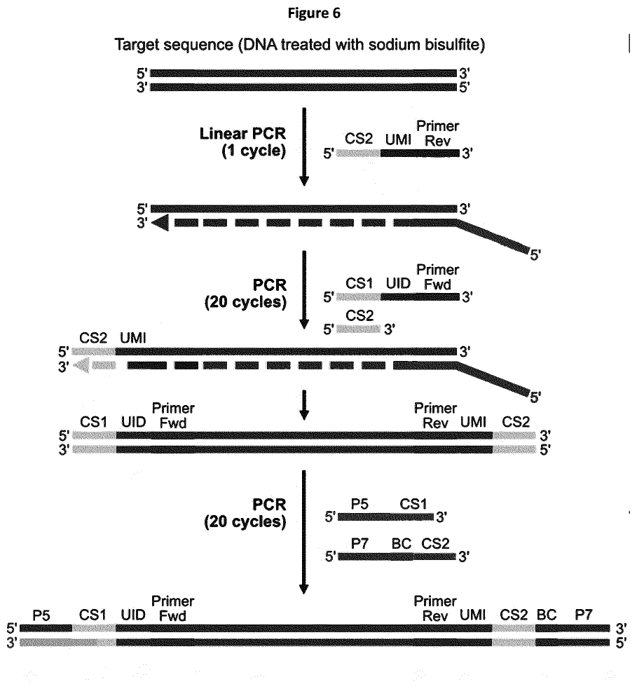

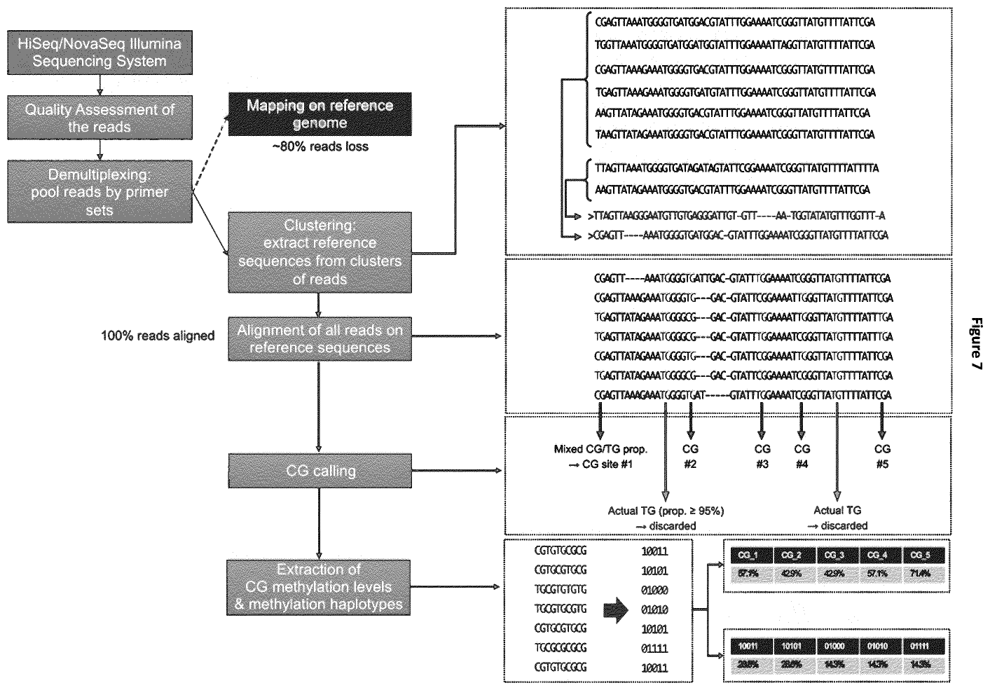

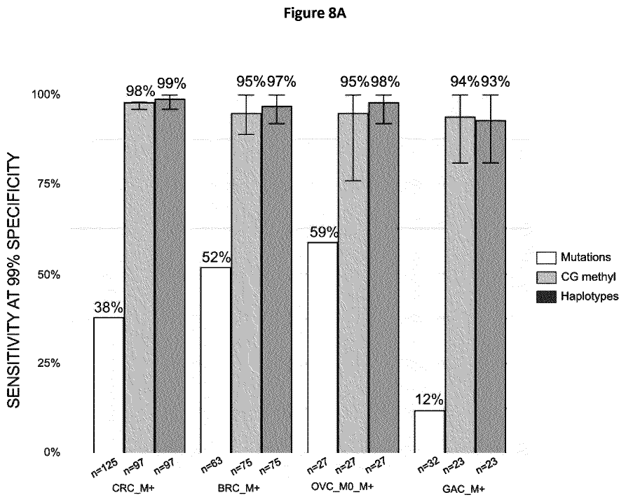

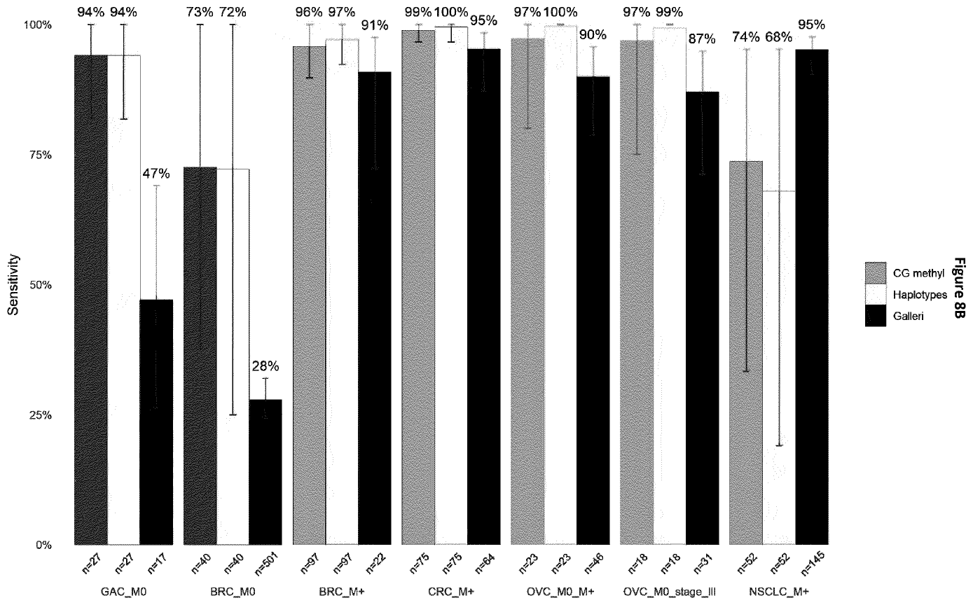

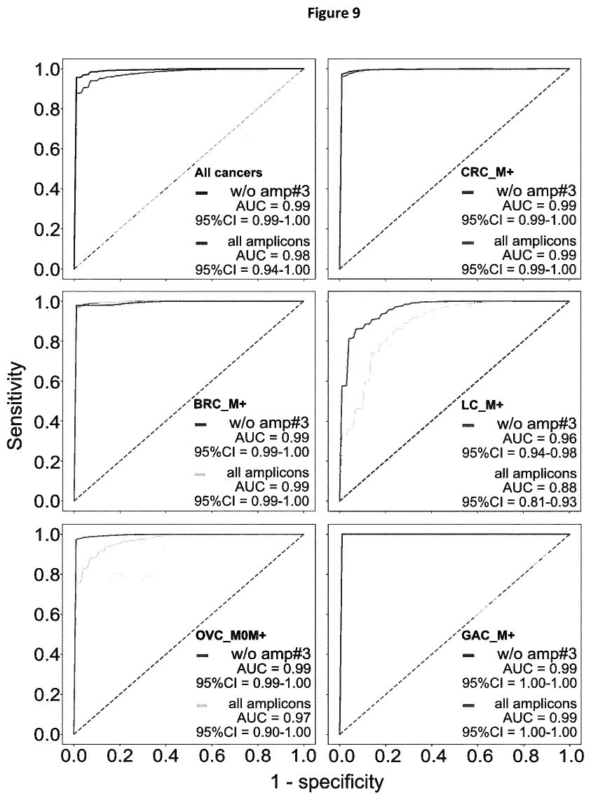

### Cancer Sample Identification Performances are Reproducible on an Independent Cohort (FIG. 4)

To validate the cfDNA L1 targeted-bisulfite-seq approach, the inventors tested a second independent cohort composed of 30 healthy donors and 160 patients affected with the same cancer types as cohort 1, except from uveal melanoma (FIG. 4A). First, when comparing the methylation patterns at each CpG sites along the L1 structure, the inventors observed a very good reproducibility overall (FIG. 4B). The inventors compared the global methylation levels excluding amplicon #3, and observed very similar distributions for each cancer types between the 2 cohorts (FIG. 4C). The inventors next tested the classifiers, trained on the first cohort, on this new set of independent samples. The inventors observed very performant classification results with an overall AUC of 0.99 (95% CI=0.99-1.00, FIG. 4D) when testing all cancers together with no annotations of the different histology. The inventors observed an improvement when removing CpG sites of amplicon 3. This is true for all classification conditions, for all cancers together or by cancer types, and in particular for lung and ovarian cancers (FIG. 9). Indeed, with the adjusted analysis, the inventors reach very high performances for each cancer type (AUC_CRC_M+=0.99; 95% C1=0.99-1.00; AUC_BRC_M+=0.99; 95% C1=0.99-1.00; AUC_NSCLC_M+=0.96; 95% C1=0.94-0.98; AUC_OVC_M0M+=0.99; 95% CI=0.99-1.00; AUC_GAC_M+=0.99; 95% CI=0.99-1.00) (FIG. 4E). The inventors further observed great sensitivities at 99% specificity and again an improvement when removing CpG sites of amplicon 3 (FIG. 4F). This demonstrates the robustness of detecting hypomethylation at L1PA element from cfDNA to detect cancer.

The Origin of the Cancer can Directly be Inferred from the L1PA Methylation Status (FIG. 5)

The inventors further analyzed if, besides the disease status, they could identify the origin of the cancer using a ‘Multiclass’ learning model. In this classification, the inventors provided the cancer type annotations for the training set and tested with multiple possible cancer classes, corresponding to each cancer type. They first tested this approach with single CpG methylation levels and only the classes which were homogeneous between cohort 1 and cohort 2, i.e. healthy donors, CRC_M+, BRC_M+ and OVC_M0M+. Remarkably, the inventors observed that the multiclass model detected the right cancer type with performances well above chance (AUC_HD=0.98 95% CI=0.94-1.00; AUC_CRC_M+=0.83 95% Cl=0.80-0.85; AUC_BRC_M+=0.72 95% CI=0.67-0.76; AUC_OVC_M0M+=0.73 95% CI=0.68-0.78) (FIG. 5A). The inventors also observed that in this case, using bootstrapping and including amplicon 3 tend to improve the performances for breast and ovarian cancers (FIG. 5A versus 5B and FIG. 5E) and also observed very similar results with haplotypes data (FIG. 5C-E). The inventors also tested the multiclass model including also the lung and gastric cancer groups (data not shown). Overall, the methylation profile of L1PA element provides information about the origin of the cancer.

Comparison with Existing Methods (FIG. 8)

Strikingly, the determination of L1PA methylation profile with the aid of the invention makes it possible to identify tumor plasmas with a substantially higher accuracy or performance than methods based on the detection of mutations. In comparison, the identification of the same tumor samples via methods used in clinics, detecting frequent recurrent mutations, does not exceed 59% for ovarian cancer (compared to 95% in the context of the present invention), 38% for colon cancer (compared to 98% in the context of the present invention), and 52% for metastatic breast cancer (compared to 95% in the context of the present invention) (FIG. 8A). The inventors also achieved remarkable performance on the cohort of 27 early gastric cancers with a detection rate of 94% as compared to 12.5% for mutation screening.

In addition, the method according to the invention reach similar or higher levels of sensitivity for 5 different cancers in comparison with the Galleri® test (Klein, E. A. et al. Annals of Oncology 32, 1167-1177 (2021)), (FIG. 8B). In contrast to the method according to the invention that targets repeated sequences such as retrotransposons, the method of Galleri® is a capture-based method targeting 100,000 uniquely mappable regions (targets which can be mapped to a specific region and only one region in the genome, i.e., in opposition to repeats target which are elements dispersed throughout the genome). In addition, the method developed by the inventors only requires to target about 82-125,000 CpG sites, that is ten times less compared to the 1,100,000 CpG sites targeted by the Galleri® test. The strongest originality and competitive edge of the method disclosed herein by inventors is to interrogate DNA methylation, in particular cfDNA methylation.

### Discussion

In this study, the inventors established a robust proof that targeting hypomethylation of transposons from cell-free DNA is a sensitive and specific biomarker to detect multiple forms of cancer non-invasively. The inventors developed a “turnkey” analysis method that identifies tumor plasmas and could quantify tumor burden. They interrogated selected repetitive regions, which provide genome-wide information, as repeats hold half of the CpG sites present in the human genome. This novel method targets hypomethylation of LINE-1 retrotransposons, which is a common feature of multiple forms of cancer, in order to capture a wide range of tumor alleles and cover the heterogeneous profiles of cancer patients in a single test. The cfDNA L1 targeted-bisulfite-seq assay could provide a substitute index of genome-wide DNA methylation levels. This allowed to generate methylation profiles from minute amounts of cfDNA, down to a few nanograms, with high precision and high coverage using an affordable sequencing depth. The inventors therefore anticipate this method to be widely applicable for the development of routine clinical tests. The strongest originality and competitive edge of this study is to interrogate cfDNA methylation at repeated sequences. Hypomethylation of repeats being common to many, if not all, cancer types, it is a promising marker for pan-cancer detection. Previous studies have left these regions aside because they are inherently difficult to map and differentially methylated regions (DMR) analysis is commonly performed on mapped data. The inventors have developed a new method to detect methylation profiles at repeats with a single base-pair resolution, without resorting to mapping on a reference genome. This allows to retain most of the produced data, which is instrumental to achieve high sensitivity and work with minute amounts of cfDNA. The results disclosed herein demonstrated high performance in detecting cancer samples and the inventors established its feasibility in six different cancer types, including three at early stages.

Overall, this assay targets about 82-125,000 CpG sites, that is ten times less compared to the 1,100,000 CpGs targeted in the existing Galleri test. However, the inventors reached similar or higher levels of sensitivity in 4 of the 5 cancers tested in common to both studies (FIG. 8B). For example, the inventors achieved a 94% sensitivity (at 99% specificity) on early stages of gastric cancers compared to 47% reached with the Galleri test. The inventors also achieved a 73% sensitivity (at 99% specificity) on early stages of breast cancers compared to only 28% achieved by Galleri (FIG. 8B). This was also the range of sensitivity reached in the CancerSeek test (<40% sensitivity for BRC_M0), which interrogated a panel of mutation within 16 genes coupled to 8 proteins (Cohen, J. D. et al. Science 1, eaar3247-10 (2018)). This highlights that breast cancer is one of the most difficult cancer to detect with liquid biopsies and that the approach according to the invention could become a game changer for early detection of breast cancer non-invasively.

The inventors were also able to demonstrate that the origin of cancers detected can be inferred from the L1PA methylation status detected in plasma DNA.

1. A method for determining a CpG methylation profile of at least one DNA sequence of interest or any fragment thereof, wherein the method comprises:
a) clustering a set of sub-sequences obtained from a DNA sequence of interest into clusters of sub-sequences;
b) selecting, for and from each cluster, one sub-sequence as a reference sequence among the sub-sequences of the cluster,
c) aligning the reference sequences of said clusters by allowing the alignment on positions of CpG dinucleotides,
d) aligning the remaining sub-sequences on selected reference sequences; and
e) determining the CpG methylation status of each sub-sequence by determining at each CpG site of the sub-sequence if the CpG dinucleotide is methylated or not, thereby determining a CpG methylation profile comprising a CpG methylation level and/or a proportion of CpG methylation haplotype of the sub-sequences,
wherein the DNA sequence of interest is or comprises a repeated sequence, said repeated sequence being distributed throughout the subject's genome, and preferably comprising high density of CpG dinucleotides;
wherein, the method optionally comprises a first step of obtaining or providing a set of sub-sequences of said DNA sequence of interest, and
wherein the method optionally comprises repeating some, or each, of steps a) through e) with other sets of sub-sequences from the DNA sequence of interest.

2. The method of claim 1, wherein the repeated sequence is a retrotransposon such as LINE, HERV, SINE, SVA, or a subfamily thereof such as in particular LINE-1, L1PA, HERV-K and Alu, or a satellite repeat such as Sat2 or Sat3 element, preferably a LINE-1 retrotransposon or any fragment or variant thereof, even more preferably a LINE-1 retrotransposon such as described under SEQ ID NO: 2 or 29 or any fragment or variant thereof.

3. A computer-implemented method of training a classifier for accurately distinguishing between a healthy CpG methylation profile and a cancerous CpG methylation profile, said method comprising:
a) providing a training set of CpG methylation profiles of DNA sequences of interest or sub-sequences thereof, said DNA sequences of interest being repeated and distributed throughout a genome and comprising high density of CpG dinucleotides, or preprocessed information obtained from said training set of CpG methylation profiles of DNA sequences of interest or sub-sequences thereof, as an input to a classifier, said training set of CpG methylation profiles comprising CpG methylation profiles of DNA sequences, or of sub-sequences thereof, from subjects identified as healthy subjects and from subjects identified as cancerous subjects; and,
b) generating an output of the classifier for each CpG methylation profile input of DNA sequence of interest or sub-sequences thereof, said output classifying the CpG methylation profile input of DNA sequence of interest or sub-sequences thereof as a healthy CpG methylation profile or as a cancerous CpG methylation profile;
wherein the CpG methylation profile comprises a CpG methylation level and/or proportion of CpG methylation haplotypes of the DNA sequence or sub-sequences thereof.

4. The method of claim 3, wherein the CpG methylation profiles of the DNA sequences of interest or sub-sequences thereof are determined by the method of claim 1.

5. An in vitro or in silico method of determining the health status of a subject, in particular of determining if the subject is a healthy subject or a subject suffering from cancer or cancer relapse, wherein the method comprises:
a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
b) using the classifier to identify the CpG methylation profile of the DNA sequence of interest or sub-sequences thereof of said subject as a healthy CpG methylation profile or as a cancerous CpG methylation profile as an output of the classifier.

6. An in vitro or in silico method of determining the origin of a tumor from a subject, wherein the method comprises:
a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile from different tumors origins, and
b) using the classifier to identify the CpG methylation profile of the DNA sequence of interest or sub-sequences thereof of said subject as a healthy CpG methylation profile or as a cancerous CpG methylation profile from a particular tumor origin to determine the origin of the tumor from the subject as an output of the classifier.

7. An in vitro or in silico method of determining the stage of a tumor from a subject, wherein the method comprises:
a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and cancerous CpG methylation profile of different stages, and
b) using the classifier to identify the CpG methylation profile of the DNA sequence of interest or sub-sequences thereof of said subject as a healthy CpG methylation profile or as a cancerous CpG methylation profile of a particular stage to determine the stage of the tumor from the subject as an output of the classifier.

8. An in vitro or in silico method of monitoring the response to an anti-cancer treatment of a subject suffering from cancer, wherein the method comprises:
a) providing at least one DNA sequence of interest or sub-sequences thereof from a first liquid biopsy from a subject suffering from cancer before the administration of the anti-cancer treatment to the subject as a first input, said DNA sequence of interest being repeated through the subject genome and comprising high density of CpG sites or a fragment thereof, or preprocessed information obtained from said first liquid biopsy, and a second liquid biopsy comprising at least one DNA sequence of interest or sub-sequences thereof from said subject after the administration of an anti-cancer treatment as a second input, or preprocessed information obtained from said second liquid biopsy, to a classifier trained to distinguish between DNA sequence having a healthy CpG methylation profile and DNA sequence having a cancerous CpG methylation profile; and
b) using the classifier to identify each CpG methylation profile of each DNA sequence of the first liquid biopsy as having a healthy CpG methylation profile or a cancerous CpG methylation profile as a first output of the classifier, and to identify each CpG methylation profile of each DNA sequence of the second liquid biopsy as having a healthy CpG methylation profile or a cancerous CpG methylation profile as a second output of the classifier, and
wherein a number of DNA sequence of interest classified as having a healthy CpG methylation profile in the second output of the classifier which is above a number of DNA sequence of interest classified as having a healthy CpG methylation profile in the first output of the classifier is indicative that the subject is responsive to said anti-cancer treatment, and
wherein a number of DNA sequence of interest classified as having a healthy CpG methylation profile in the second output of the classifier which is equal to or below a number of DNA sequence of interest classified as having a healthy CpG methylation profile in the first output of the classifier is indicative that the subject does not respond to said anti-cancer treatment.

9. An in vitro or in silico method of assessing the potency of a compound to revert a cancerous CpG methylation profile of a DNA sequence of interest from a subject suffering from cancer into a healthy CpG methylation profile, wherein the method comprises:
a) providing DNA sequences of interest or sub-sequences thereof from the subject having been treated with a compound, said DNA sequences of interest being repeated and distributed throughout the subject's genome and comprising high density of CpG dinucleotides or any fragment thereof, or preprocessed information obtained from said at least one DNA sequence of interest or sub-sequences thereof, as an input to a classifier trained to distinguish between DNA sequences having a healthy CpG methylation profile or a cancerous CpG methylation profile; and
b) using the classifier to detect DNA sequences having a healthy CpG methylation profile and/or DNA sequences having a cancerous CpG methylation profile as an output of the classifier, wherein an amount of DNA sequences having a healthy methylation profile above a reference amount of DNA sequences having a healthy methylation profile obtained from the subject before any treatment with the compound is indicative that the compound is able to revert the cancerous CpG methylation profile into a healthy CpG methylation profile.

10. An in vitro or in silico method of predicting the ability of a compound to treat a cancer comprising assessing the potency of a compound to revert a cancerous CpG methylation profile of a DNA sequence of interest from a subject into a healthy CpG methylation profile according to claim 9,
wherein an amount of DNA sequences classified as having a healthy CpG methylation profile, which is above the reference amount is indicative that said compound is useful in the treatment of said cancer.

11. The method of claim 5, wherein the CpG methylation profiles of the DNA sequence of interest or sub-sequence thereof is determined by
a) clustering a set of sub-sequences obtained from the DNA sequence of interest into clusters of sub-sequences;
b) selecting, for and from each cluster, one sub-sequence as a reference sequence among the sub-sequences of the cluster,
c) aligning the reference sequences of said clusters by allowing the alignment on positions of CpG dinucleotides,
d) aligning the remaining sub-sequences on selected reference sequences; and
e) determining the CpG methylation status of each sub-sequence by determining at each CpG site of the sub-sequence if the CpG dinucleotide is methylated or not, thereby determining a CpG methylation profile comprising a CpG methylation level and/or a proportion of CpG methylation haplotype of the sub-sequences,
wherein the DNA sequence of interest is or comprises a repeated sequence, said repeated sequence being distributed throughout the subject's genome, and preferably comprising high density of CpG dinucleotides;
wherein, the method optionally comprises a first step of obtaining or providing a set of sub-sequences of said DNA sequence of interest, and
wherein the method optionally comprises repeating some, or each, of steps a) through e) with other sets of sub-sequences from the DNA sequence of interest.

12. The method of claim 5, wherein the classifier is trained by
a) providing a training set of CpG methylation profiles of DNA sequences of interest or sub-sequences thereof, said DNA sequences of interest being repeated and distributed throughout a genome and comprising high density of CpG dinucleotides, or preprocessed information obtained from said training set of CpG methylation profiles of DNA sequences of interest or sub-sequences thereof, as an input to a classifier, said training set of CpG methylation profiles comprising CpG methylation profiles of DNA sequences, or of sub-sequences thereof, from subjects identified as healthy subjects and from subjects identified as cancerous subjects; and,
b) generating an output of the classifier for each CpG methylation profile input of DNA sequence of interest or sub-sequences thereof, said output classifying the CpG methylation profile input of DNA sequence of interest or sub-sequences thereof as a healthy CpG methylation profile or as a cancerous CpG methylation profile;
wherein the CpG methylation profile comprises a CpG methylation level and/or proportion of CpG methylation haplotypes of the DNA sequence or sub-sequences thereof.

13. The method of any one of claim 1, wherein the DNA sequence of interest is a circulating cell-free DNA (cfDNA) sequence.

14. A computing system comprising:
a memory storing at least one instruction of a classifier trained according to the method of claim 3, and
a processor accessing the memory for reading the aforesaid instruction(s) and executing the method by
a) providing a DNA sequence of interest or sub-sequences thereof from the subject, or preprocessed information obtained from said DNA sequence or sub-sequences, said DNA sequence of interest being a DNA sequence encoding a repeated sequence distributed throughout the subject's genome and comprising high density of CpG dinucleotides as an input to a classifier trained to distinguish between a healthy CpG methylation profile and a cancerous CpG methylation profile, and
b) using the classifier to identify the CpG methylation profile of the DNA sequence of interest or sub-sequences thereof of said subject as a healthy CpG methylation profile or as a cancerous CpG methylation profile as an output of the classifier.

15. A kit of primers or probes targeting sub-sequences of a DNA sequence encoding a LINE-1 retrotransposon having a sequence as set forth in SEQ ID NO: 2 or 29, said kit comprising at least 4 primers or probes selected from the group of primers or probes having a sequence as set forth in SEQ ID NO: 11, 12, 13, 14, 15, 16, 17, 18, 19, 20, 21, 22, 23, 24, 25 or 26 respectively or a sequence having at least 85% identity thereto.

16. A method for the diagnosis of cancer comprising using the kit according to claim 15 for amplifying sub-sequences of a DNA sequence encoding a LINE-1 retrotransposon having a sequence set forth as SEQ ID NO: 2 or 29, wherein the cancer is selected from the group consisting of colon cancer, breast cancer, lung cancer, uveal melanoma cancer, ovary cancer and stomach cancer.

17. The method of claim 6, wherein the CpG methylation profiles of the DNA sequence of interest or sub-sequence thereof is determined by the method of claim 1.

18. The method of claim 7, wherein the CpG methylation profiles of the DNA sequence of interest or sub-sequence thereof is determined by the method of claim 1.

19. The method of claim 8, wherein the CpG methylation profiles of the DNA sequence of interest or sub-sequence thereof is determined by the method of claim 1.

20. The method of claim 9, wherein the CpG methylation profiles of the DNA sequence of interest or sub-sequence thereof is determined by the method of claim 1.

21. The method of claim 10, wherein the CpG methylation profiles of the DNA sequence of interest or sub-sequence thereof is determined by the method of claim 1.

22. The method of claim 6, wherein the classifier is trained according to the method of claim 3.

23. The method of claim 7, wherein the classifier is trained according to the method of claim 3.

24. The method of claim 8, wherein the classifier is trained according to the method of claim 3.

25. The method of claim 9, wherein the classifier is trained according to the method of claim 3.

26. The method of claim 10, wherein the classifier is trained according to the method of claim 3.

27. A computing system comprising:
a memory storing at least one instruction of a classifier trained according to the method of claim 3, and
a processor for accessing the memory for reading the aforesaid instruction(s) and executing the method according to claim 6.

28. A computing system comprising:
a memory storing at least one instruction of a classifier trained according to the method of claim 3, and
a processor for accessing the memory for reading the aforesaid instruction(s) and executing the method according to claim 7.

29. A computing system comprising:
a memory storing at least one instruction of a classifier trained according to the method of claim 3, and
a processor for accessing the memory for reading the aforesaid instruction(s) and executing the method according to claim 8.

30. A computing system comprising:
a memory storing at least one instruction of a classifier trained according to the method of claim 3, and
a processor for accessing the memory for reading the aforesaid instruction(s) and executing the method according to claim 9.

31. A computing system comprising:
a memory storing at least one instruction of a classifier trained according to the method of claim 3, and
a processor for accessing the memory for reading the aforesaid instruction(s) and executing the method according to claim 10.
